(19) United States
(12) Patent Application Publication
(10) Pub. No.: US 2019/0161754 A1
(43) Pub. Date: May 30, 2019

(54) FLUORESCENT BIOSENSOR FOR 2', 3'-CGAMP

(71) Applicant: The Regents of the University of California, Oakland, CA (US)

(72) Inventors: Ming C. Hammond, Berkeley, CA (US); Yichi Su, Berkeley, CA (US); Debojit Bose, Berkeley, CA (US)

(21) Appl. No.: 16/098,316

(22) PCT Filed: Jun. 9, 2017
(86) PCT No.: PCT/US17/36859

§ 371 Date: Nov. 1, 2018

Related U.S. Application Data
(60) Provisional application No. 62/349,556, filed on Jun. 13, 2016.

(51) Int. Cl. C12N 15/113; C12Q 1/6825

(52) U.S. Cl. CPC C12N 15/113; C12N 2310/16; C12Q 1/6825

(57) ABSTRACT

A single stranded nucleic acid biosensor for 2′, 3′-cGAMP is provided. The single stranded nucleic acid may include a 2′, 3′-cGAMP-binding riboswitch domain comprising a transducer stem and a dye-binding aptamer domain that is operably connected to the transducer stem. A 2′, 3′-cGAMP-binding riboswitch domain. The dye-binding aptamer domain can be a Spinach2 aptamer. The 2′, 3′-cGAMP biosensor may further include a signaling chromophore specifically bound to the Spinach2 aptamer domain, where the sensor is configured to fluorescently activate the signaling chromophore upon specific binding of 2′, 3′-cGAMP to the 2′, 3′-cGAMP-binding riboswitch domain. Also provided are methods in which the subject 2′, 3′-cGAMP biosensors find use including methods for determining the level of cGAS activity in a sample or a cell. Nucleic acid constructs and host cells including the same are also provided.

### CROSS-REFERENCE

This application claims the benefit of U.S. Provisional Patent Application No. 62/349,556, filed Jun. 13, 2016, which application is incorporated herein by reference in its entirety.

### STATEMENT REGARDING FEDERALLY SPONSORED RESEARCH

This invention was made with government support under Grant No. OD008677 awarded by the National Institutes of Health. The government has certain rights in the invention.

### INTRODUCTION

The cGAS-cGAMP-STING pathway is an important immune surveillance pathway which gets activated in presence of cytoplasmic DNA either due to microbial infection or patho-physiological condition, including cancer and autoimmune disorder. Cyclic GMP-AMP synthase (cGAS) belongs to the nucleotidyltransferase family and is a universal DNA sensor that is activated upon binding to cytosolic dsDNA to produce the signaling molecule (2′-5′, 3′-5′) cyclic GMP-AMP (or 2′, 3′-cGAMP or Cyclic guanosine monophosphate-adenosine monophosphate). Acting as a second messenger during microbial infection, 2′, 3′-cGAMP binds and activates STING, leading to production of type I interferon (IFN) and other co-stimulatory molecules that trigger the immune response. Besides its role in infectious disease, the cGAS/STING pathway has emerged as a promising new target for cancer immunotherapy and autoimmune diseases. DNA fragments present in the tumor microenvironment are proposed to activate cGAS in dendritic cells (DC), followed by IFN-induced DC maturation and activation of a potent and beneficial immune response against cancer cells. In a separate context, dysregulation of the cGAS/STING pathway has been implicated in self DNA triggered inflammatory and autoimmune disorders, such as systemic lupus erythematosus (SLE) and Aicardi-Goutieres syndrome.

It is of interest to explore the regulation and activation status of the cGAS/STING pathway. Deconvoluting the role of the cGAS/STING pathway in immune responses is made difficult by the presence of multiple surveillance pathways that trigger the same IFN signal downstream. For example, one of the most commonly used and sensitive methods to detect activation of the cGAS/STING pathway in vivo is a luciferase reporter fused to an IFN-β promoter. However, other DNA sensors (e.g. TLR9 and IFI16), RNA sensors (e.g. RIG, MDA5) and immune modulators also activate expression of this reporter as all of them activate downstream IFN production. Additionally, the assay requires transfection of the reporter DNA, and this foreign DNA can activate cGAS and other DNA sensing pathways, which can mask the underlying physiology. A direct method of detecting and quantifying 2′, 3′-cGAMP rather than downstream signals is of interest for diagnostic purposes and for distinguishing cGAS/STING from other nucleic acid sensing pathways, in order to determine its contribution to the overall immune response.

### SUMMARY

A single stranded nucleic acid biosensor for 2′, 3′-cGAMP is provided. The single stranded nucleic acid may include a 2′, 3′-cGAMP-binding riboswitch domain comprising a transducer stem and a dye-binding aptamer domain that is operably connected to the transducer stem of the 2′, 3′-cGAMP-binding riboswitch domain. The 2′, 3′-cGAMP-binding riboswitch domain can be a GEMM-II riboswitch domain. The dye-binding aptamer domain can be a Spinach2 aptamer. The 2′, 3′-cGAMP biosensor may further include a signaling chromophore specifically bound to the Spinach2 aptamer domain, where the sensor is configured to fluorescently activate the signaling chromophore upon specific binding of 2′, 3′-cGAMP to the 2′, 3′-cGAMP-binding riboswitch domain. Also provided are methods in which the subject 2′, 3′-cGAMP biosensors find use including methods for determining the level of cGAS activity in a sample and methods for determining the level of cGAS activity in a cell. Nucleic acid constructs for the single stranded nucleic acid and host cells including the same are also provided.

### BRIEF DESCRIPTION OF THE DRAWINGS

It is understood that the drawings, described below, are for illustration purposes only. The drawings are not intended to limit the scope of the present teachings in any way.

FIG. 1 depicts illustrates the structures of c-di-GMP, 3′, 3′-cGAMP and 2′, 3′-cGAMP.

FIG. 2 depicts secondary structure of GEMM-II riboswitch-based biosensor that detects 2′, 3′-cGAMP (SEQ ID NO: 17). Mutations of interest are boxed in black in the structure.

FIG. 3 shows in vitro fluorescence activation and binding affinity measurements for RNA-based biosensors with 2′, 3′-cGAMP.

FIG. 4 shows a rational mutagenesis analysis of the Bh P1-6 biosensor.

FIG. 5 depicts hydrogen bond interaction of the native GEMM-II riboswitch with G_{α} and G_{β} of c-di-GMP and hypothetical interaction of G103A biosensor with adenine in the 2′, 3′-cGAMP.

FIG. 6 shows Bh P1-6 G103A biosensor detects 2′, 3′-cGAMP produced in the cGAS enzyme reaction (black). Enzyme reactions that lack one component served as negative controls (white).

FIG. 7 illustrates in situ detection of cGAS activity by RNA-based fluorescent biosensor.

FIG. 8 shows the Z′ scores determined in a high throughput cGAS activity assay using an exemplary RNA-based biosensor in 384-well format.

FIG. 9 illustrates performance of a RNA-based fluorescent biosensor for assaying cGAS inhibition by nucleic acid intercalator quinacrine (QC).

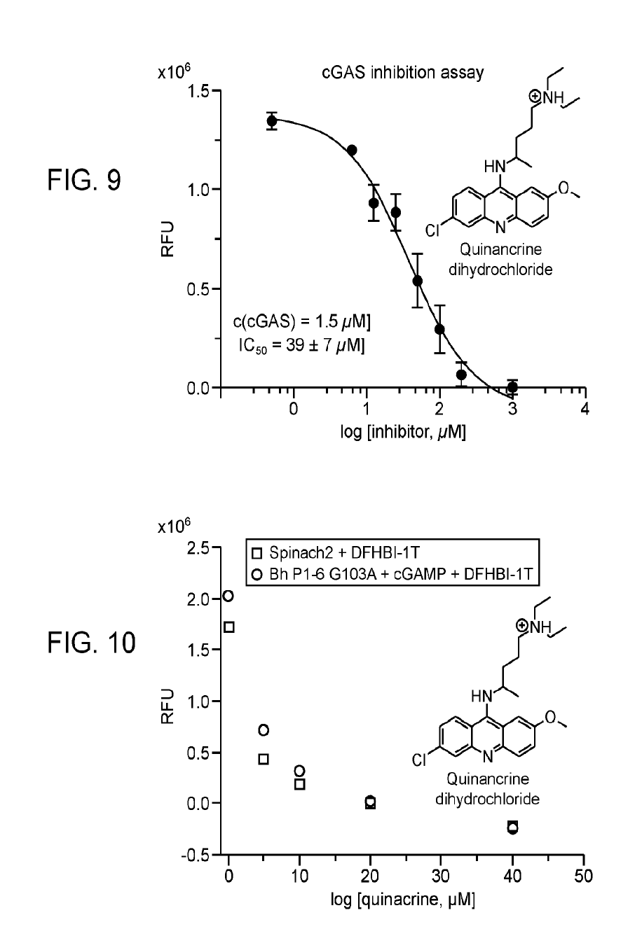

FIG. 10 illustrates dose-dependent loss of fluorescence in presence of QC for both the 2′, 3′-cGAMP biosensor and Spinach2 aptamer.

FIG. 11 shows results of TLC assay for cGAS inhibition by various nucleic acid intercalators by quinacrine (QC), ethidium bromide (EtBr), actinomycin D (AD) and methylene blue (MB).

FIG. 12, panels A-B, shows RNA-based fluorescent biosensors for detection of 2′, 3′-cGAMP, c-di-GMP and 3′, 3′-cGAMP production in enzyme-expressing bacteria. (A) Representative flow cytometry analysis of live E. coli expressing plasmids encoding both biosensor and cyclic dinucleotide synthases after incubation in media containing DFHBI-1T. Fluorescence values on x-axis are shown using HyperLog scaling. The average mean fluorescence intensity (MFI) from two independent biological replicates are shown in the bar graph, error bars depict standard deviations. (B) In vitro fluorescence biosensor-based analysis of cyclic dinucleotide content of cell extracts from E. coli expressing the corresponding cyclic dinucleotide synthases. Error bars depict standard deviations for two independent biological replicates.

FIG. 13 illustrates a schematic of a procedure for detection of 2′, 3′-cGAMP in mammalian cell extracts using a RNA-based biosensor.

FIG. 14, panels A-C, shows results of assays performed according to FIG. 13 for detection of 2′, 3′-cGAMP in mammalian cell extracts. (Panels A and B) Fluorescence readings for different concentrations of L929 (B) or HEK293T (C) cell extracts (DNA- or mock-stimulated). (Panel C) Fluorescence readings for L929 cell extracts (DNA- or mock-stimulated) treated with active or denatured SVPD prior to biosensor addition.

FIG. 15 shows a standard curve of fluorescence readings from mock-stimulated L929 cell extracts doped with known concentrations of 2′, 3′-cGAMP (black circles). Average fluorescence reading from DNA-stimulated L929 cell extracts is shown in open circle. Error bars indicate standard deviations for two independent biological replicates. P-values are calculated from student's t-test comparison: **, P<0.01; ***, P<0.001, n.s, not significant, P>0.05. Linear regression R²=0.98. [2′,3′-cGAMP] in DNA-stimulated L929 extracts=1.5 uM.

FIG. 16, panels A-B, illustrate a comparison of interactions between (A) GEMM-I riboswitch or (B) GEMM-II riboswitch with 2′-hydroxyls and phosphodiester oxygens of c-di-GMP. The interactions are shown in red dashes.

FIG. 17, panels A-B, illustrate a screen for GEMM-II riboswitch-based 2′,3′-cGAMP biosensor. Panel A shows a phylogenetic screen for GEMM-II riboswitch-based biosensor. Error-bars represent the standard deviation of 3 independent replicates. Panel B shoes the fluorescent detection profiles of the biosensor hits from first round screen against c-di-GMP, 3′, 3′-cGAMP and 2′, 3′-cGAMP.

FIG. 18 illustrates selectivity profiling of both Bh P1-5 delC WT and G103A biosensors against bacterial metabolites, c-di-GMP and 2′, 3′-cGAMP.

FIG. 19, panels A-B, illustrate a TLC based analysis of human cGAS cyclic dinucleotide product (2′, 3′-cGAMP) formation. Reaction products were labeled with indicated α-³²P. Reactions were treated with or without calf intestinal alkaline phosphatase (CIP) and analyzed by thin layer chromatography. Image in panel A is representative of multiple independent experiments. Panel B shows the analysis of cGAS inhibition by quinacrine (QC) via RNA-based fluorescent biosensor. The determined IC₅₀ was revealed to be artefactual due to the intercalating effect of QC to the RNA based biosensor.

FIG. 20, panels A-C, illustrate LC-MS analysis of 2′,3′-cGAMP production in E. coli. Panel A shows LC-MS analysis of E. coli cell extract expressing plasmids encoding cGAS. 675 m/z trace showed strong spectra in the time range between 4 to 10 min, corresponding to the production of large amount of 2′, 3′-cGAMP, while 691 m/z trace (corresponding to c-di-GMP) was negative. Panels B and C illustrate quantitation of 2′,3′-cGAMP in E. coli cell extract. Varied concentrations of 2′,3′-cGAMP were assayed on an Agilent 1260 Infinity LC-MS system equipped with a Poroshell 120 C-18 column, with 260 nm absorbance (Panel B) and mass spec signal (Panel C) shown. Standard curve of quantifying 2′,3′-cGAMP based on the integration of mass spec signal was shown in inset in (Panel C).

FIG. 21 shows data regarding the basal 2′, 3′-cGAMP level in L929 cells. Different concentrations of non-stimulated L929 cell extracts were treated with either active or inactive SVPD. SVPD treatment did not cause any change in fluorescent intensity, indicating the basal 2′, 3′-cGAMP level (if any) is below current detection limit.

FIG. 22 shows the origin of background fluorescence in non-stimulated cell extract was investigated. Auto-fluorescence from the cell extract, non-specific activation of DFHBI fluorescence and background fluorescence of the biosensor with DFHBI were analyzed and compared. −, cell extract alone; +, cell extract+DFHBI; ++, cell extract+biosensor+DFHBI.

FIG. 23, Panel A shows the efficacy of heat inactivation on SVPD was tested using Spinach2 RNA aptamer. SVPD is shown to degrade RNA aptamer (gray) and heat inactivation with optimized conditions appear to be efficient in inactivating SVPD (black). No decrease in fluorescence signal of Spinach2 was observed in presence of heat inactivated SVPD compared to no SVPD control. Panel B demonstrates a significant reduction in 2′, 3′-cGAMP-responsive biosensor fluorescence was observed in presence of active SVPD (black), while heat inactivated SVPD remained ineffective (gray). This indicated SVPD is efficient in degrading 2′, 3′-cGAMP under assay condition.

### DEFINITIONS

Before describing exemplary embodiments in greater detail, the following definitions are set forth to illustrate and define the meaning and scope of the terms used in the description. Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

As used herein, the term “aptamer” refers to a nucleic acid molecule, such as RNA or DNA (in some cases RNA), that is capable of binding to a specific molecule with high affinity and specificity (Ellington et al., Nature 346: 818-22, 1990; and Tuerk et al., Science 249: 505-10, 1990). Although aptamer in general can bind a wide variety of exemplary ligands, including, without limitation, small molecules, such as drugs, metabolites, intermediates, cofactors, transition state analogs, ions, metals, nucleic acids, polysaccharides, glycoproteins, hormones, receptors and cell surfaces (such as cell walls and cell membranes), and toxins, aptamers that may be used in the present disclosure bind target molecules such as fluorophores and 2′, 3′-cGAMP.

As used herein, the term stem refers to secondary structural feature of a single stranded nucleic acid that includes base pairing. The stem-loop structure in which a base-paired helix ends in a short unpaired loop is a common feature and is a building block for larger structural motifs, such as cloverleaf structures, which are four-helix junctions such as those found in transfer RNA. Internal loops (a short series of unpaired bases in a longer paired helix) and bulges (regions in which one strand of a helix has “extra” inserted bases with no counterparts in the opposite strand) are also frequent. A pseudoknot is a nucleic acid secondary structure containing at least two stem-loop structures in which half of one stem is intercalated between the two halves of another stem. Pseudoknots fold into knot-shaped three-dimensional conformations but are not true topological knots. The base pairing in pseudoknots is not well nested; that is, base pairs occur that “overlap” one another in sequence position.

The terms “specific binding,” “specifically binds,” and the like, refer to the ability of one binding moiety to preferentially bind directly to a target molecule relative to other molecules or moieties in a sample. In certain embodiments, the affinity between a given binding moiety and the molecule or moiety to which it specifically binds when they are specifically bound to each other in a binding complex is characterized by a K_{D} (dissociation constant) of 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, or 10⁻¹⁵ M or less (it is noted that these values can apply to any specific binding pair interactions mentioned elsewhere in this description, in certain embodiments).

As used herein, the terms “nucleic acid”, “oligonucleotide” and “polynucleotide” are used interchangeably and refer to a polymer of 2 or more nucleotides, such as a DNA sequence or analog thereof, or an RNA sequence or analog thereof. Nucleic acids are formed from nucleotides, including, but not limited to, the nucleotides listed herein. In some instances, the terms “nucleic acid molecule” and “polynucleotide” refer to deoxyribonucleotides or ribonucleotides and polymers thereof in either single- or double-stranded form. The term “RNA” refers to ribonucleic acid, in some cases, in single-stranded form. Unless specifically limited, the terms encompass nucleic acids/RNAs containing known analogues of natural nucleotides which have similar binding properties as the reference nucleic acid and are metabolized in a manner similar to naturally occurring nucleotides. The terms may also encompass nucleic acids/RNAs containing chemical modifications, such as modifications at the base moiety, sugar moiety, and/or phosphate backbone that tend to increase stability or half-life of the molecules in vivo. For example, these molecules may have naturally occurring phosphodiester linkages, as well as those having non-naturally occurring linkages, e.g., for stabilization purposes, or for enhancing hydrophobic interaction with protein ligands.

As used herein, the terms “riboswitch” and “riboswitch domain” refers to a regulatory segment of a messenger RNA that is capable of binding to a target molecule, e.g., a small molecule, and is involved in regulating the production of a protein encoded by the mRNA.

“Isolated” or “purified” generally refers to isolation of a substance (compound, polynucleotide, protein) such that the substance comprises the majority percent of the sample in which it resides. In some cases, in a sample a substantially purified component comprises 50% or more, such as 80%-85% or more, or 90-95% or more of the sample. Techniques for purifying polynucleotides and polypeptides of interest are well-known in the art and include, for example, ion-exchange chromatography, affinity chromatography and sedimentation according to density.

“Nucleotide” refers to naturally- and non-naturally-occurring nucleotides and nucleotide analogs. Nucleotides include, but are not limited to, adenosine, cytosine, guanosine, thymidine, uracil, 4-acetylcytosine, 8-hydroxy-N-6-methyladenosine, aziridinyl-cytosine, pseudoisocytosine, 5-(carboxyhydroxylmethyl) uracil, 5-fluorouracil, 5-bromouracil, 5-carboxymethylaminomethyl-2-thiouracil, 5-carboxy-methylaminomethyluracil, dihydrouracil, inosine, N6-iso-pentenyladenine, 1-methyladenine, 1-methylpseudouracil, 1-methylguanine, 1-methylinosine, 2,2-dimethyl-guanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonyl-methyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine and 2,6-diaminopurine, 6-mercaptopurine, 5-fluorouracil, 5-iodo-2′-deoxyuridine and 6-thioguanine, cytosine exocyclic amines, substitution of 5-bromo-uracil, backbone modifications, methylations, and unusual base-pairing combinations. Additional analogs include at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil; beta-D-mannosylqueosine, 5-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, and 2,6-diaminopurine.

Exemplary modified base moiety may be selected from the group including, but not limited to: 5-fluorouracil, 5-bromouracil, 5-chlorouracil, 5-iodouracil, hypoxanthine, xanthine, 4-acetylcytosine, 5-(carboxyhydroxytriethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil; beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl)uracil, (acp3)w, and 2,6-diaminopurine.

Exemplary modified sugar moiety may be selected from the group including but not limited to arabinose, 2-fluoroarabinose, xylulose, and hexose.

Exemplary neutral peptide-like backbone modification include: peptide nucleic acid (PNA) (see, e.g., in Perry-O'Keefe et al. (1996) Proc. Natl. Acad. Sci. USA 93:14670 and in Eglom et al. (1993) Nature 365:566), or modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The nucleic acid may be in any physical form, e.g., linear, circular, or supercoiled.

As used herein, the term “domain” refers to a continuous or discontinuous sequence of amino acid residues or nucleotides. As used herein, the term “region” refers to a continuous sequence of amino acid residues or nucleotides.

“Complementary” refers to a nucleotide or polynucleotide sequence that hybridizes to a given nucleotide or polynucleotide sequence. For instance, for DNA, the nucleotide A is complementary to T, and vice versa, and the nucleotide C is complementary to G, and vice versa. For instance, in RNA, the nucleotide A is complementary to the nucleotide U, and vice versa, and the nucleotide C is complementary to the nucleotide G, and vice versa. Complementary nucleotides include those that undergo Watson and Crick base pairing and those that base pair in alternative modes. For instance, as used herein for RNA, the nucleotide G is complementary to the nucleotide U and vice versa, and the nucleotide A is complementary to the nucleotide G and vice versa. Therefore, in an RNA molecule, the complementary base pairs are A and U, G and C, G and U, and A and G. Other combinations, e.g., A and C, A and A, G and G, or C and U, are considered to be non-complementary base pairs.

A “complementary sequence” comprises individual nucleotides that are complementary to the individual nucleotides of a given sequence, where the complementary nucleotides are ordered such that they will pair sequentially with the nucleotides of the given sequence. Such a complementary sequence is said to be the “complement” of the given sequence.

As used herein, the terms “linker”, “linkage” or “linking group” refer to a linking moiety that connects two groups. In some instances, the linker may have a backbone of 100 atoms or less in length, e.g., 50 atoms or less in length, including 20 atoms or less in length. A linker may be a covalent bond that connects two groups or a chain of between 1 and 20 atoms in length, for example of about 1, 2, 3, 4, 5, 6, 8, 10, 12, 14, 16, 18 or 20 carbon atoms in length, where the linker may be linear, branched, cyclic or a single atom. In certain cases, one, two, three, four or five or more carbon atoms of a linker backbone may be optionally substituted with a sulfur, nitrogen or oxygen heteroatom. The bonds between backbone atoms may be saturated or unsaturated. In some instances, no more than one, two, or three unsaturated bonds will be present in a linker backbone. The linker may include one or more substituent groups, for example an alkyl, aryl or alkenyl group. A linker may include, without limitations, oligo(ethylene glycol), ethers, thioethers, tertiary amines, amino acid residues, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone.

In certain embodiments, the linking group comprises 1-15 carbon atoms and/or 0-6 heteroatoms. In certain embodiments, the linking group is selected from the group consisting of —(CH₂)n-C(O)—, or —C(O)(CH₂)n- or —C(O)(CH₂)n-NHC(O)—, or —C(O)(CH2)n-NHC(O)(CH₂)n-, or —(CH₂)nSCH₂C(O)—, or —(CH₂)n-C(O)NH—(CH2)n-, or —(CH₂)n-NH—C(O)—, or —(CH₂)n-NH—C(O)—(CH₂)n-, or —C(O)—(CH₂)n-, or —(CH₂)n-NH—; and n is an integer from 1 to 10, and including acid salts thereof. In certain embodiments, the linking group is —(CH₂)n-C(O)NH—(CH₂)n-, where each n is an integer from one to ten. In certain embodiments, the linking group is —(CH₂)n-C(O)NH—(CH₂)n-, where each n is one or two. In certain embodiments, the linking group is —(CH₂)n-, where n is an integer from one to ten. In certain embodiments, the linking group is —(CH₂)—. In certain embodiments, the linking group is —(CH₂)n-C(O)N(CH₂)n(CH₃)—(CH₂)n-, where each n is an integer from one to ten. In certain embodiments, the linking group is —(CH₂)n-C(O)N(CH₂)n(CH₃)—(CH₂)n-, where each n is one or two. In certain embodiments, the linking group is —(CH₂)n-C(O)N(CH₃)—(CH₂)n-, where each n is an integer from one to ten. In certain embodiments, the linking group is —(CH₂)n-C(O)N(CH₃)—(CH₂)n, where each n is one or two. In certain embodiments, the linking group comprises 10-15 carbon atoms and/or 0-6 heteroatoms. Additionally, linkers can comprise modified or unmodified nucleotides, nucleosides, polymers, sugars and other carbohydrates, polyethers, such as for example, polyethylene glycols, polyalcohols, polypropylenes, propylene glycols, mixtures of ethylene and propylene glycols, polyalkylamines, polyamines such as spermidine, polyesters such as poly(ethyl acrylate), polyphosphodiesters, and alkylenes.

A linker may be cleavable or non-cleavable. As used herein, the term “cleavable linker” refers to a linker that can be selectively cleaved to produce two products. Application of suitable cleavage conditions to a molecule containing a cleavable linker that is cleaved by the cleavage conditions will produce two cleavage products. A cleavable linker may be stable, e.g. to physiological conditions, until it is contacted with a stimulus capable of cleaving the cleavable linker.

The term “fluorophore” refers to a molecule that, when excited with light having a selected wavelength, emits light of a different wavelength, which may emit light immediately or with a delay after excitation. Fluorophores, include, without limitation, fluorescein dyes, e.g., 5-carboxyfluorescein (5-FAM), 6-carboxyfluorescein (6-FAM), 2′,4′,1,4,-tetrachlorofluorescein (TET), 2′,4′, 5′,7′,1,4-hexachlorofluorescein (HEX), and 2′,7′-dimethoxy-4′,5′-dichloro-6-carboxyfluorescein (JOE); cyanine dyes, e.g. Cy3, CYS, Cy5.5, QUASARTM dyes etc.; dansyl derivatives; rhodamine dyes e. g. 6-carboxytetramethylrhodamine (TAMRA), CAL FLUOR dyes, tetrapropano-6-carboxyrhodamine (ROX). BODIPY fluorophores, ALEXA dyes, Oregon Green, pyrene, perylene, benzopyrene, squarine dyes, coumarin dyes, luminescent transition metal and lanthanide complexes and the like. The term fluorophore includes excimers and exciplexes of such dyes.

Other terms used herein and in the claims adopt their plain meanings as would have been understood by one of skill in the relevant art, that are not inconsistent with the usages in the instant specification. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques of described herein are those well known and commonly used in the art.

### DETAILED DESCRIPTION

Before the various embodiments are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a biosensor” includes a plurality of such biosensors and reference to “the biosensor” includes reference to one or more biosensor and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Single Stranded Nucleic Acid Aptamer

Aspects of the present disclosure include a single stranded nucleic acid having a 2′, 3′-cGAMP-binding riboswitch domain and a signaling chromophore-binding Spinach2 aptamer domain where the two domains are operably connected via a transducer stem. Any convenient aptamers and aptamer domains may be adapted for use in the subject biosensors. As used herein, by “operably connected” is meant the two aptamer domains are integrated into a single stranded nucleic acid, e.g., via a transducer stem located at or near to both the 2′, 3′-cGAMP and signaling chromophore binding sites, such that binding of the 2′, 3′-cGAMP target molecule to the 2′, 3′-cGAMP-binding riboswitch domain may induce a conformational change to modulate (e.g., enhance) binding of the signaling chromophore to the Spinach2 aptamer domain to produce a detectable signal (e.g., a fluorescence enhancement).

The present disclosure provides a strategy for operably connecting a 2′, 3′-cGAMP-binding GEMM-II riboswitch domain and a Spinach2 aptamer domain that includes connecting the two domains via an internal paired stem region that integrates the P1′ region of the GEMM-II riboswitch domain and the P2 region of the Spinach2 aptamer domain. This strategy provides for proximity of the 2′, 3′-cGAMP and signaling chromophore binding sites in the integrated nucleic acid that are capable of interaction upon ligand binding, e.g., spatially, sterically, and/or via a change in structure of the ligand-bound complex. Aspects of the present disclosure include modified riboswitch domains that include modifications, relative to a natural parent riboswitch domain, such as a shortened P1 stem or mutation(s) within the binding region, that provide for more selective binding to 2′,3′-cGAMP versus the natural ligand (e.g., cyclic di-GMP).

Single stranded nucleic acids, such as aptamers, may fold to form a variety of complex secondary structures and are capable of specifically binding a target molecule. An aptamer may be obtained by in vitro or in vivo selection for binding of a target molecule (e.g., a 2′, 3′-cGAMP ligand). Single stranded nucleic acids of interest from which the subject sensors may be designed or derived include naturally occurring sequences, such as naturally occurring aptamers or riboswitches. Aptamers may be developed to bind particular ligands by employing known in vivo or in vitro (in some cases, in vitro) selection techniques known as SELEX (Ellington et al., Nature 346: 818-22, 1990; and Tuerk et al., Science 249, 505-10, 1990). Methods of making aptamers are also described in, for example, US-2009-0082217-A1, U.S. Pat. No. 5,582,981, PCT Publication No. WO 00/20040, U.S. Pat. No. 5,270,163, Lorsch and Szostak, Biochemistry, 33:973 (1994), Mannironi et al., Biochemistry 36:9726 (1997), Blind, Proc. Nat'l. Acad. Sci. USA 96:3606-3610 (1999), Huizenga and Szostak, Biochemistry, 34:656-665 (1995), PCT Publication Nos. WO 99/54506, WO 99/27133, WO 97/42317 and U.S. Pat. No. 5,756,291.

Aptamers have specific binding regions which are capable of forming complexes with an intended target molecule in an environment wherein other substances in the same environment are not complexed to the nucleic acid. The specificity of the binding is defined in terms of the comparative dissociation constants (K_{d}) of the aptamer for its ligand as compared to the dissociation constant of the aptamer for other materials in the environment or unrelated molecules in general. A ligand is one which binds to the aptamer with greater affinity than to unrelated material. In some cases, the K_{d} for the aptamer with respect to its ligand will be at least about 10-fold less than the K_{d} for the aptamer with unrelated material or accompanying material in the environment. In certain cases, the K_{d} will be at least 50-fold less, such as at least about 100-fold less, e.g., at least 200-fold less. An aptamer will in some cases be between 10 and 300 nucleotides in length. In certain instances, an aptamer will be between 30 and 100 nucleotides in length.

In some cases, the subject biosensor includes a nucleic acid having a GEMM-II riboswitch domain that specifically binds 2′, 3′-cGAMP and a Spinach2 aptamer domain for binding a signaling chromophore (e.g., a fluorophore such as DFHBI or an analog thereof). In some instances, the GEMM-II riboswitch domain and the Spinach2 aptamer domain are operably connected to each other such that binding of 2′, 3′-cGAMP to the riboswitch domain activates the fluorescence of a DFHBI fluorophore bound to the Spinach2 aptamer domain.

2′, 3′-cGAMP-Binding Riboswitch Domain

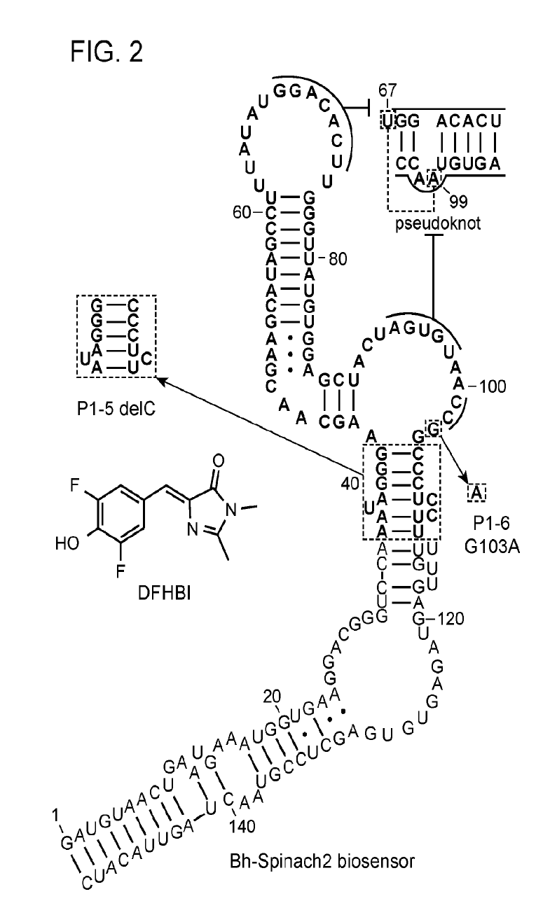

Any convenient riboswitch domains may be adapted for use in the subject nucleic acids and biosensors. By “adapted for use” is meant that a parent riboswitch of interest may be modified according to the strategy described herein (see e.g., FIG. 2) to produce a subject single stranded nucleic acid for use as a biosensor for specifically binding 2′, 3′-cGAMP. 2′, 3′-cGAMP-binding riboswitch domains of interest which may be modified according to the strategies and methods described herein include, but are not limited to, those nucleic acids described by Lee et al. ((2010). An allosteric self-splicing ribozyme triggered by a bacterial second messenger. Science 329, 845-848), the disclosure of which is herein incorporated by reference. In some embodiments, the 2′, 3′-cGAMP-binding riboswitch domain of interest is derived from one of the riboswitch domain sequences (SEQ ID NOs: 1-16 and 18-25) shown in Table 1. In some embodiments, the 2′, 3′-cGAMP-binding riboswitch domain of interest is derived from the riboswitch domain sequence (SEQ ID NO: 17) shown in FIG. 2.

In certain instances, the 2′, 3′-cGAMP-binding riboswitch domain is not derived from a GEMM-I riboswitch domain. In certain instances, the 2′, 3′-cGAMP-binding riboswitch domain selectively binds 2′, 3′-cGAMP in the presence of cyclic di-GMP. By selectively binds is meant the 2′, 3′-cGAMP-binding riboswitch domain has an apparent affinity for 2′, 3′-cGAMP that is 2 fold stronger or more than it has for cyclic di-GMP, such as 5-fold stronger or more, 10-fold or more, 30 fold stronger or more, 100-fold stronger or more.

In some embodiments, the 2′, 3′-cGAMP-binding riboswitch domain comprises a sequence having 80% or greater sequence identity (e.g., 85% or greater, 90% or greater, or 95% or greater sequence identity) to a sequence having one of SEQ ID NO:1-23 but excluding that portion of the riboswitch domain which is removed during Spinach2 aptamer domain integration as described herein. In some embodiments, the spinach2 aptamer domain comprises a 5′-terminal domain region and a discontinuous 3′-terminal domain region that together define the Spinach2 aptamer secondary structure, see e.g., FIG. 2, which is capable of binding dye DFHBI. In some embodiments, the 2′, 3′-cGAMP-domain comprises a continuous region connected to the Spinach2 aptamer at the 5′- and 3′ terminals which come together at a transducer stem region (see e.g., FIG. 2). The continuous region of the 2′, 3′-cGAMP-binding riboswitch domain defines a 2′, 3′-cGAMP binding site in the pseudo-knot riboswitch secondary structure that provides for specific binding of 2′, 3′-cGAMP. In some cases, the riboswitch and Spinach2 aptamer domains can be connected via a transducer stem region of connecting sequences.

The 2′, 3′-cGAMP-binding riboswitch domain can include a domain that is modified by comparison to a natural GEMM-II domain sequence of interest. Such modifications can be included to provide for binding to 2′,3′-cGAMP or to increase selectivity for 2′,3′-cGAMP versus other cyclic dinucleotide compounds (e.g., cyclic di-GMP). In certain instances, the 2′, 3′-cGAMP-binding riboswitch domain is derived from a GEMM-II riboswitch domain.

In some embodiments, a consensus sequence motif for a GEMM-II riboswitch structure of interest is: NNNNNRG A RRC NNUGAN NYRYNNNY U (N)₁₋₃ AYNU GGRCACYU (N)₁₋₂₂ RNNNRYRN (N)₁₋₂ GA GYY AGU RGUGCAACC GR CYNNNNN (SEQ ID NO:26), where marked residues (bold, italics and/or underlined) denote some structural features, such as regions that contain complementary base-pairs to form a stem P1 (NNNNNRG and CYNNNNN), P2 (RRC and GYY), P3 (NYRYNNNY and RNNNRYRN), pseudoknot base pairing (GGRCACYU and RGUGCAACC), N denotes any nucleotide, R denotes purines (A or G), and Y denotes pyrimidines (C or U).

In some instance, a Bacillus halodurans (Bh) sequence, following this pattern, has a sequence as follows:

(SEQ ID NO: 27)
AAUAGGG A [symbol] AACGAA GCAUAGCC U UU AUAU GGACACUU G
GGUUAUGU G GA [symbol] ACU AGUGUAACC GG CCCUCCUU

where marked residues (bold, italics and/or underlined) denote some structural features, such as P1 (AAUAGGG and CCCUCCUU), P2 (AGC and GCU), P3 (GCAUAGCC and GGUUAUGU), pseudoknot base pairing (GGACACUU and AGUGUAACC).

In certain instances, the GEMM-II sequence (or 2′,3′-cGAMP binding riboswitch) is: ARRCNNUGANNYRYNNNYUUUAYNUGGRCACYUGGGUUAUGUGGAGYYAGURGU GCAACCGR (SEQ ID NO:28), with a P1 stem of 4 or 5 or 6 base pairs. In certain cases, the sequence comprises the following structural properties:

A P2 stem:
5′-RRC-3′
3′-YYG-3′
A P3 stem:
5′-NYRYNNNY-3′
3′-NRYRNNNR-5′
A pseudoknot base pairing:
5′-GGRCACYU-3′
3′-CCAACGUGR-5′
A transducer (P1) stem:
5′-NNNNNRG-3′
3′-NNNNNYC-3′

Also, a G to A mutation is optionally included, with the sequence (see e.g., G to A denoted in bold and underlined in the sequence): ARRCNNUGANNYRYNNNYUUUAYNUGGRCACYUGGGUUAUGUGGAGYYAGURGU GCAACCAR (SEQ ID NO:29). In certain cases, the mutation is present. In certain instances, the mutation is absent.

In any of the subject sequences depicted above, it is understood that various regions of the single stranded sequence may not be fully base-paired, see e.g., the exemplary Bh sequence.

### Spinach2 Aptamer Domain

As used herein, the term “Spinach2 aptamer domain” refers to a signaling chromophore-binding aptamer domain comprising a P1 stem region and a P2 stem region connected via a signaling chromophore-binding site comprising a cyclic sequence of nucleotides. In some instances, the P1 region is an open region which includes 5′- and 3′ terminals, e.g., at the end of a stem region. An exemplary Spinach2 aptamer structure is represented in FIG. 2 which demonstrates the structural features of the subject Spinach2 aptamer domain. Spinach2 aptamer domains of interest which may adapted for use in the subject methods and domains include, but are not limited to those described by Jaffrey et al. in U.S. Pat. No. 9,664,676, the disclosure of which is herein incorporated by reference in its entirety and by Song et al. (Song, W., Strack, R. L., Svensen, N., and Jaffrey, S. R. (2014). Plug-and-play fluorophores extend the spectral properties of Spinach. J Am Chem Soc 136, 1198-1201). By “open P1 stem” region is meant that one end of the P1 stem region is terminated by a 3′ and 5′ terminals and the other end of the P1 stem is connected to the cyclic sequence defining the signaling chromophore-binding site. The P1 stem region may include 3′ and 5′ sequences defining 15-20 base pairs (e.g. 15, 16, 17, 18 19 or 20 base pairs) and may include in total 35-55 nucleotides, such as 40-55, 45-55, 48-52 or 49 nucleotides.

By “open P2 stem region” is meant that one end of the P2 stem has 3′ and 5′ termini and the other end of the P2 stem is connected to the cyclic sequence binding site. The 3′ and 5′ termini of the P2 stem can be connected to the riboswitch domain and become a part of a stem region connecting the two domains. The P2 stem may be truncated in length (e.g., as described herein) relative to a parent Spinach2 aptamer. In some cases, the P2 stem has 5 or less base pairs, although not every nucleotide in the stem need by paired to a nucleotide in the opposing strand of the stem (e.g., a bulging nucleotide). The P2 stem may include 15 or fewer nucleotides in total. The signaling chromophore-binding site may comprise a discontinuous and cyclic sequence of nucleotides that includes a total of 30 nucleotides or less, such as 25 nucleotides or less, such as 17-21 nucleotides (e.g., 17, 18, 19, 20 or 21 nucleotides). FIG. 2 depicts one exemplary discontinuous cyclic sequence of nucleotides comprising a first sequence of 8 nucleotides (e.g., 5′-AGGACGGG-3′) and a second sequence of 11 nucleotides (e.g., 5′-GUAGAGUGUGA-3′) (SEQ ID NO:30) that together make up a discontinuous cyclic sequence which defines the signaling chromophore binding site.

Any convenient signaling chromophore-binding aptamers may be adapted for use (e.g., as described in FIG. 2) in the subject single stranded nucleic acids. Signalling chromophore-binding aptamers which may be adapted for use in the subject single stranded nucleic acid aptamers include, but are not limited to, Spinach2 aptamers described by Paige et al., Science 2011, 333, 642-646; Nawrocki et al., Nucleic Acids Research 2015, 43, D130-137; Strack et al. Nature Methods, 10, 1219-1224 (2013); Jaffrey et al., U.S. Publication No. 2014/0220560; and Krishnan et al., WO2015/033237, the disclosures of which are herein incorporated by reference. In some instances, the Spinach2 aptamer domain is derived from a Spinach2 aptamer that has been modified according to FIG. 2 (e.g., connected to a riboswitch domain via a transducer stem). In certain instances, the Spinach2 aptamer domain may optionally include one or more additional looped stem regions extending from the signaling chromophore binding site.

In some cases, a consensus sequence motif for a GEMM-II riboswitch structure of interest comprises the following sequences:

a 5′ sequence of Spinach2 RNA aptamer sequence:

(SEQ ID NO: 31)
5′-GAUGUAACUGAAUGAAAUGGUGAAGGACGGGUCCA-3′

a 3′ sequence of Spinach2 RNA aptamer sequence:

(SEQ ID NO: 32)
5′-UUGUUGAGUAGAGUGUGAGCUCCGUAACUAGUUACAUC-3′

a 2′,3′-cGAMP binding domain comprising the following sequence:

(SEQ ID NO: 33)
5′-ARRCNNUGANNYRYNNNYUUUAYNUGGRCACYUGGGUUAUGUGGAGY
YAGURGUGCAACCGR-3′;

and a transducer stem sequence (e.g., as described herein).

In some instances, this sequence comprises the following structural properties:

A P2 stem:
5′-RRC-3′
3′-YYG-3′
A P3 stem:
5′-NYRYNNNY-3′
3′-NRYRNNNR-5′
A pseudoknot base pairing:
5′-GGRCACYU-3′
3′-CCAACGUGR-5′
A transducer (P1) stem:
5′-NNNNNRG-3′
3′-NNNNNYC-3′

Also, a G to A mutation is optionally included, with the sequence (G to A highlighted in the sequence):

(SEQ ID NO: 34)
ARRCNNUGANNYRYNNNYUUUAYNUGGRCACYUGGGUUAUGUGGAGYYAG
URGUGCAACCAR.

### Signalling Chromophore

The Spinach2 aptamer domain may specifically bind any convenient signaling chromophore to provide for a fluorescent signal upon binding to 2′, 3′-cGAMP. Any convenient signaling chromophores that bind to a Spinach or Spinach2 aptamer domain may be utilized in the subject biosensors. In some instances, the signaling chromophore is a fluorophore. In some cases, the signaling chromophore is a fluorogenic compound, such that the chromophore has no significant fluorescence when it is not bound to the Spinach2 aptamer. The signaling chromophore may be switched to a fluorescent state of interest by binding to a Spinach2 aptamer domain. For example, binding of a nucleic acid molecule (e.g., as described herein) to the signaling chromophore substantially enhances fluorescence of the compound upon exposure to radiation of suitable wavelength. In some cases, the signaling chromophore is switched to a fluorescent state of interest by a 2′, 3′-cGAMP ligand-induced conformational change of the subject biosensor.

In certain embodiments, the signaling chromophore is a 4-hydroxybenzlidene imidazolinone (HBI) or derivative thereof, such as a chromophore described by Paige et al, Science, Vol. 333 no. 6042 pp. 642-646, 2011. In certain embodiments, the signaling chromophore is selected from one of the following:

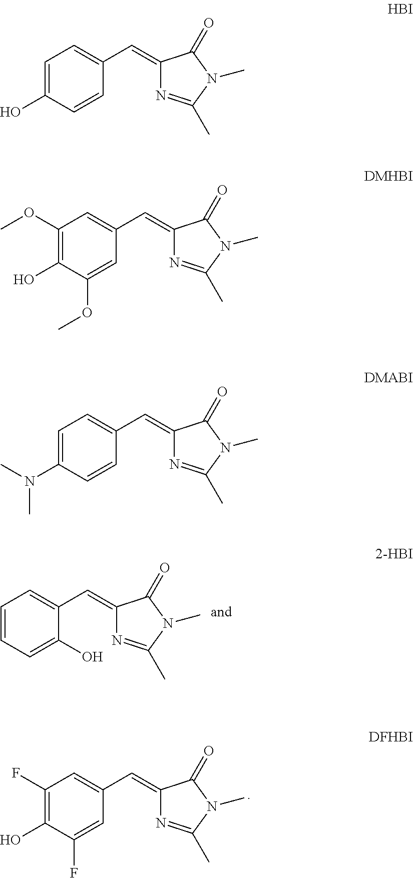

In certain embodiments, the signaling chromophore is DFHBI-IT.

Signaling chromophores of interest that may be utilized in the subject biosensors for specifically binding to the Spinach2 aptamer domain include, but are not limited to, DFHBI, (Z)-4-(3,5-difluoro-4-hydroxybenzylidene)-2-methyl-1-(2,2,2-trifluoroethyl)-1H-imidazol-5(4H)-one (DFHBI-1T); (Z)-4-(3,5-difluoro-4-hydroxybenzylidene)-1-methyl-2-(trifluoromethyl)-1H-imidazol-5(4H)-one (DFHBI-2T); hydroxyl analogs of DFHBI (e.g., DFHBI-1HO, (Z)-4-(3,5-difluoro-4-hydroxy-benzylidene)-1-hydroxy-2-methyl-1H-imidazol-5(4H)-one); and methoxyl analogs of DFHBI (e.g., DFHBI-1MO, (Z)-4-(3,5-difluoro-4-hydroxybenzylidene)-1-methoxy-2-methyl-1H-imidazol-5(4H)-one); and fluorophores described by Song et al., J Am Chem Soc. 2014 Jan. 29; 136(4): 1198-1201. In some instances, the Spinach2 aptamer domain is capable of specifically binding (Z)-4-(3,5-difluoro-4-hydroxybenzylidene)-1,2-dimethyl-1H-imidazol-5(4H)-one (DFHBI).

In some embodiments, the signaling chromophore is a compound comprising a methyne bridge between a substituted aromatic ring system and a substituted imidazol(thi)one, oxazol(thi)one, pyrrolin(thi)one, or furan(thi)one ring, wherein binding of a nucleic acid molecule (e.g., as described herein) to the compound substantially enhances fluorescence of the compound upon exposure to radiation of suitable wavelength, such as a compound described by Jaffrey et al. in US Publication No. 20120252699, the disclosure of which is herein incorporated by reference. In certain embodiments, the signaling chromophore is a compound according to formula (I)

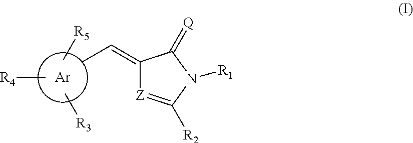

wherein, Q is S or O, Y is O or N, Z is N or C(H), Ar is an aromatic or hetero-aromatic ring system comprising one or two rings; R₁ is present when Y is N, and is a C₁₋₈ hydrocarbon or —(CH₂)ₙ—R₆ where n is an integer greater than or equal to 1; R₂ is methyl, a mono-, di-, or tri-halo methyl, oxime, O-methyl-oxime, imine, substituted or unsubstituted phenyl with up to three substituents (R₇-R₉), C₂₋₈ unsaturated hydrocarbon optionally terminated with an amine, amide, carboxylic acid, (meth)acrylate, ester, enone, oxime, O-methyl-oxime, imine, nitromethane, nitrile, ketone, mono-, di-, tri-halo, nitro, cyano, acrylonitrile, acrylonitrile-enoate, acrylonitrile-carboxylate, acrylonitrile-amide, or a second aromatic or hetero-aromatic ring; R₃-R₅ are independently selected from H, hydroxy, alkyl, alkoxy, fluoro, chloro, bromo, amino, alkylamino, dialkylamino, alkylthio, cyano, mercapto, nitro, and mono-, di-, or tri-halo methyl, ketone, and carboxylic acid; R₆ is H, a surface-reactive group, a solid surface, or a functional group that can be linked to a surface-reactive group or solid surface; R₇-R₉ are independently selected from H, hydroxy, alkyl, alkoxy, fluoro, chloro, bromo, amino, alkylamino, dialkylamino, alkylthio, cyano, mercapto, nitro, and mono-, di-, or tri-halo methyl, ketone, and carboxylic acid; and salts thereof;

In some instances, the signaling chromophore is a compound of formula (I) wherein: (i) R₃-R₅ cannot all be H; (ii) when R₁ and R₂ are methyl, and R₄ and R₅ are H, R₃ is not hydroxy, methoxy, or dimethylamino; and (iii) when R₁ is methyl, R₄ and R₅ are H, and R₃ is hydroxy, R₂ is not a conjugated hydrocarbon chain.

### Transducer Stem

The 2′, 3′-cGAMP-binding domain and the Spinach2 aptamer domain may be connected as described herein by integration of a stem of the 2′, 3′-cGAMP-binding domain with the P2 stem of the Spinach2 aptamer domain. The stems may be integrated and connected in a variety of ways to provide a transducer stem capable of operably connecting the 2′, 3′-cGAMP-binding site with the signaling transducer binding site. In some cases, the resulting integrated transducer stem comprises 10 base pairs or less, such as 9 base pairs or less, 8 base pairs or less, 7 base pairs or less, 6 base pairs or less or 5 base pairs or less (e.g., 4 or 5 base pairs, NNNN or NNNNN, where N is any convenient nucleotide), where additional optional bulging nucleotides may also be included. FIG. 2 illustrates a transducer stem region of interest. In some cases, the transducer stem is 4-6 base pairs, and may optionally comprise an unpaired nucleotide. In certain cases, the transducer stem is composed of 2 strands, each independently 4-8 nucleotides in length.

In certain embodiments of a riboswitch aptamer sequence of interest, the connecting transducer stem is a P1 stem of 5 base pair length with bulges removed (P1-5delC).

In certain instances, the connecting transducer stem is a P1-5 delC stem. In some embodiments, the connecting transducer stem includes the following pair of sequences:

5′-AAUAGGG-3′;
and
3′-UUCCUCCC-5′.

In some embodiments, the connecting transducer stem includes the following pair of sequences:

5′-AUAGGG-3′;
and
3′-UCUCCC-5′.

### Biosensor

Aspects of the present disclosure include biosensors for 2′, 3′-cGAMP. The biosensor may include a single stranded nucleic acid (e.g., as described herein); and a signaling chromophore (e.g., as described herein) specifically bound to the Spinach2 aptamer domain. In some cases, the single stranded nucleic acid includes a 2′, 3′-cGAMP-binding riboswitch domain; and a Spinach2 aptamer domain including a P2 stem that is operably connected to the 2′, 3′-cGAMP-binding riboswitch domain. In certain instances, the connecting stem comprises 10 base pairs or less. The sensor is configured to fluorescently activate the signaling chromophore upon specific binding of 2′, 3′-cGAMP to the 2′, 3′-cGAMP-binding riboswitch domain, e.g., via a ligand activated conformational change. In some instances, the signaling chromophore is a DFHBI fluorophore.

The subject biosensors may be modified in a variety of ways depending on the application in which the biosensor finds use. Moieties of interest suitable for adapting for use in modifying the nucleic acid component of the subject biosensors include, but are not limited to, any convenient moiety suitable for attachment to the 3′ or 5′ terminal of a nucleic acid, a protein domain, a polypeptide, a peptide tag, a specific binding moiety, a polymeric moiety such as a polyethylene glycol (PEG), a carbohydrate, a dextran or a polyacrylate, a linker, a chemoselective functional group, a moiety that imparts desirable drug-like properties, a detectable label (e.g., a fluorophore), a support, a half-life extending moiety, a fatty acid, a solid support and a linker.

In some instances, the single stranded nucleic acid of the biosensor is attached to a solid support, via an optional linker. Any convenient solid supports may be utilized, including but not limited to, a bead, a microarray, a flat surface, a chromatography support, etc.

### Constructs

Nucleic acid molecules of the present disclosure (e.g., RNA) can be delivered to target cells in vitro or in vivo. A number of methods have been developed for delivering nucleic acids into cells; e.g., they can be injected directly into the tissue site, or modified nucleic acids, designed to target the desired cells can be administered systematically. Another approach utilizes a recombinant DNA construct in which the RNA or other biosensor nucleic acid is placed under the control of a strong pol III or pol II promoter. The use of such a construct to transfect target cells will result in the transcription of sufficient amounts of the subject RNA. For example, a vector or expression construct can be such that it is taken up by a target cell and directs the transcription of a subject RNA. Such a vector or expression construct can remain episomal or become chromosomally integrated, as long as it can be transcribed to produce the desired product. Such vectors can be constructed by recombinant DNA technology methods standard in the art. Vectors can be plasmid, viral, or others known in the art, used for replication and expression in mammalian cells. A promoter may be operably linked to the sequence encoding the subject RNA. Expression of the subject encoded sequences can be by any promoter known in the art to act in mammalian, in some cases, human cells. Such promoters can be inducible or constitutive. Such promoters include but are not limited to: the SV40 early promoter region (Bernoist and Chambon, Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc. Natl. Acad. Sci. USA 78:1441-1445 (1981)), the regulatory sequences of the metallothionein gene (Brinster et al, Nature 296:3942 (1982)), etc. Any type of plasmid, cosmid, YAC or viral vector can be used to prepare the recombinant DNA construct that can be introduced directly into the sample or tissue site. Alternatively, viral vectors can be used which selectively infect the desired sample or tissue, in which case administration may be accomplished by another route (e.g., systematically).

Also provided is an expression vector or construct having a coding sequence that is transcribed to produce one or more transcriptional products that produce a subject nucleic acid of interest in the treated cells. Any convenient expression vectors appropriate for producing an aptamer-regulated nucleic acid may be utilized. For example, the expression vector is selected from an episomal expression vector, an integrative expression vector, and a viral expression vector.

In certain embodiments, the expression vector can be designed to include one or more subject nucleic acid or transcript, such as in the 3′ untranslated region (3′-UTR), so as to regulate transcription, stability and/or translation of a RNA transcript in a manner dependent on a ligand. To further illustrate, the expression construct can include a coding sequence for a polypeptide such that the mRNA transcript includes both the polypeptide coding sequence as well as one or more of the RNA of the invention. In this way, expression of the construct can be controlled dependent on the ligand(s) to which the aptamer(s) bind.

### Host Cells

Aspects of the invention include providing a biosensor, or nucleic acid component thereof, in a host cell. The cells that are provided with the biosensor or nucleic acid component thereof may include a cGAS enzyme of interest. The cell that is provided with the biosensor may vary depending on the specific application being performed. Target cells of interest include eukaryotic cells, e.g., animal cells, where specific types of animal cells include, but are not limited to: insect, worm or mammalian cells. Various mammalian cells may be used, including, by way of example, equine, bovine, ovine, canine, feline, murine, non-human primate and human cells. Among the various species, various types of cells may be used, such as hematopoietic, neural, glial, mesenchymal, cutaneous, mucosal, stromal, muscle (including smooth muscle cells), spleen, reticulo-endothelial, epithelial, endothelial, hepatic, kidney, gastrointestinal, pulmonary, fibroblast, and other cell types. Hematopoietic cells of interest include any of the nucleated cells which may be involved with the erythroid, lymphoid or myelomonocytic lineages, as well as myoblasts and fibroblasts. Also of interest are stem and progenitor cells, such as hematopoietic, neural, stromal, muscle, hepatic, pulmonary, gastrointestinal and mesenchymal stem cells, such as ES cells, epi-ES cells and induced pluripotent stem cells (iPS cells).

In some embodiments, these cells are cells that have been engineered to include the nucleic acid component of a subject biosensor. The protocol by which the cells are engineered to include the desired nucleic acid may vary depending on one or more different considerations, such as the nature of the target cell, the nature of the biosensor, etc. The cell may include expression constructs having coding sequences for the single stranded nucleic acid component of the subject biosensor under the control of a suitable promoter. The coding sequences will vary depending on the particular nature of the nucleic acid encoded thereby, and in some cases will include at least a first domain that encodes the 2′, 3′-cGAMP-binding riboswitch domain and a second domain that encodes a Spinach2 aptamer domain. The two domains may be joined directly or linked to each other by a linking domain, e.g., a transducer stem. The domains encoding the nucleic acid are in operational combination, i.e., operably linked, with requisite transcriptional mediation or regulatory element(s). Requisite transcriptional mediation elements that may be present in the expression module include promoters (including tissue specific promoters), enhancers, termination and polyadenylation signal elements, splicing signal elements, and the like. Of interest in some instances are inducible expression systems. The various expression constructs in the cell may be chromosomally integrated or maintained episomally, as desired. Accordingly, in some instances the expression constructs are chromosomally integrated in a cell. Alternatively, one or more of the expression constructs may be episomally maintained, as desired. In yet other embodiments, the nucleic acid of interest may be provided via microinjection of mRNA or proteins. The cells may be prepared using any convenient protocol, where the protocol may vary depending on nature of the cell, the location of the cell, e.g., in vitro or in vivo, etc. Where desired, vectors, such as viral vectors, may be employed to engineer the cell to express the chimeric proteins as desired. Protocols of interest include those described in published PCT application WO1999/041258, the disclosure of which protocols are herein incorporated by reference.

As desired, cells may be engineered in vitro or in vivo. For target cells that are engineered in vitro, such cells may ultimately be introduced into a host organism. Depending upon the nature of the cells, the cells may be introduced into a host organism, e.g. a mammal, in a wide variety of ways. In some instances, the cell comprising the biosensor system(s) is part of a multicellular organism, e.g., a transgenic animals or animal comprising a graft of such cells that comprise a biosensor system(s). Any convenient methods for generating cells having targeted gene modifications through homologous recombination may be utilized. For various techniques for transfecting mammalian cells, see Keown et al., (1990), Meth. Enzymol. 185:527-537.

### Methods

As summarized above, aspects of the present disclosure include methods for determining the level of 2′, 3′-cGAMP in a sample. As such, aspects of the method include, contacting the sample with a biosensor, e.g., as described above, under conditions in which 2′, 3′-cGAMP, if present in the sample, specifically binds to the biosensor to activate fluorescence of a bound signaling chromophore (e.g., fluorophore). The detected fluorescent signal may then be used to determine the level, e.g., concentration, of 2′, 3′-cGAMP in the sample. Any convenient controls and standards may be utilized in determining the level of 2′, 3′-cGAMP in the sample.

In some instances, the fluorescence activation of the signaling chromophore is an increase in fluorescence of 10% or more, such as, 20% or more, 30% or more, 40% or more, 50% or more, 60% or more, 70% or more, 80% or more, 90% or more, 100% or more, 120% or more, 150% or more, 200% or more, 300% or more, 500% or more, or even more relative to the fluorescence of the biosensor when it is not bound to 2′, 3′-cGAMP. In some instances, the fluorescence activation of the signaling chromophore is by 40% or more.

In some cases, the biosensor is configured to specifically bind 2′, 3′-cGAMP, e.g., with an affinity for 2′, 3′-cGAMP that is at least 10-fold stronger affinity as compared to a control analyte of interest, such as at least 30-fold stronger affinity, at least 100-fold stronger affinity, at least 300-fold stronger affinity, at least 1000-fold stronger affinity, e.g., as measured by a dose response curve.

In some instances, the subject biosensor activates fluorescence of the bound signaling chromophore when bound to 2′, 3′-cGAMP with a response that is at least 10 fold more sensitive to 2′, 3′-cGAMP than other analytes of interest, such as at least 30 fold more sensitive, at least 100 fold more sensitive, at least 300-fold more sensitive, at least 1000-fold more sensitive, or even more sensitive to 2′, 3′-cGAMP than other analytes of interest, e.g., as measured by a dose response curse. Based on the response of the biosensor, a level of 2′, 3′-cGAMP in the sample may be determined. In some cases, the determined level of 2′, 3′-cGAMP is independent of the level of other analytes of interest. cGAS enzymes of interest produce 2′, 3′-cGAMP, and as such, the level of 2′, 3′-cGAMP in the sample is dependent, at least in part, on the cGAS activity in the cell. In certain embodiments, the subject method further includes determining a cGAS activity of the sample based on the determined level of 2′, 3′-cGAMP.

Any convenient protocol for contacting the sample with the biosensor may be employed. The particular protocol that is employed may vary, e.g., depending on whether the sample is in vitro or in vivo. For in vitro protocols, contact of the sample with the biosensor may be achieved using any convenient protocol. In some instances, the sample includes cells that are maintained in a suitable culture medium, and the biosensor is introduced into the culture medium. In certain in vitro protocols, no cells are present and the biosensor is simply contacted with other components (e.g., proteins, etc.) of the desired protocol in a convenient container, e.g., vial. For in vivo protocols, any convenient administration protocol may be employed. Depending upon the binding affinity of the biosensor, the response desired, the manner of administration, e.g. intravenous, subcutaneous, intraperitoneal, oral, intramuscular, etc., the half-life, the number of cells present, various protocols may be employed.

The term “sample” as used herein relates to a material or mixture of materials, in some cases, in fluid form, containing one or more components of interest. Samples may be derived from a variety of sources such as from food stuffs, environmental materials, a biological sample or solid, such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components). The term “sample” may also refer to a “biological sample”. As used herein, the term “a biological sample” refers to a whole organism or a subset of its tissues, cells or component parts (e.g. body fluids, including, but not limited to, blood, mucus, lymphatic fluid, synovial fluid, cerebrospinal fluid, saliva, amniotic fluid, amniotic cord blood, urine, vaginal fluid and semen). A “biological sample” can also refer to a homogenate, lysate or extract prepared from a whole organism or a subset of its tissues, cells or component parts, or a fraction or portion thereof, including but not limited to, plasma, serum, spinal fluid, lymph fluid, the external sections of the skin, respiratory, intestinal, and genitourinary tracts, tears, saliva, milk, blood cells, tumors and organs. In certain embodiments, the sample has been removed from an animal or plant. Biological samples may include cells. The term “cells” is used in its conventional sense to refer to the basic structural unit of living organisms, both eukaryotic and prokaryotic, having at least a nucleus and a cell membrane. In certain embodiments, cells include prokaryotic cells, such as from bacteria. In other embodiments, cells include eukaryotic cells, such as cells obtained from biological samples from animals, plants or fungi. In certain embodiments, the sample includes a cell extract. In some cases of the subject methods, the sample includes extracted cell lysates.

Components in a sample are termed “analytes” herein. In many embodiments, the sample is a complex sample containing at least about 10², 5×10², 10³, 5×10³, 10⁴, 5×10⁴, 10⁵, 5×10⁵, 10⁶, 5×10⁶, 10⁷, 5×10⁷, 10⁸, 10⁹, 10¹⁰, 10¹¹, 10¹² or more species of analyte.

The subject methods may further include evaluating the sample for appearance of a detectable signal. Evaluation of the sample may be performed using any convenient method, and at any convenient time before, during or after application of a biosensor to the sample. Evaluation of the sample may be performed continuously, or by sampling at one or more time points during the subject method. Aspects of the subject methods include detecting fluorescence from the biosensor thereby determining the level of 2′, 3′-cGAMP in the sample. Detecting fluorescence may include exciting the biosensor with one or more lasers at an interrogation point of the sample, and subsequently detecting fluorescence emission from the signaling chromophore using one or more optical detectors.

Also provided by the present disclosure is a method for determining level of methyltransferase activity in a cell. Aspects of the method include contacting the cell with a single stranded nucleic acid (e.g., as described herein) and a signaling chromophore (e.g., as described herein) to produce a 2′, 3′-cGAMP biosensor in situ. In certain instances, contacting the cell with the subject nucleic acid includes expressing a construct encoding the subject nucleic acid (e.g., as described herein). In some instances, contacting the cell with the subject nucleic acid includes introducing a composition including the subject nucleic acid to the cell.

In some instances, the method is a method including detecting fluorescence from the signaling chromophore (e.g., a DFHBI fluorophore) of the 2′, 3′-cGAMP biosensor thereby determining the level of an enzyme activity in the sample, where the enzyme either utilizes 2′, 3′-cGAMP as a substrate or co-substrate or produces 2′, 3′-cGAMP as a product (cGAS). In certain cases, the sample is a cellular sample.

Aspects of the method include detecting fluorescence from the signaling chromophore (e.g., a DFHBI fluorophore) of the 2′, 3′-cGAMP biosensor thereby determining the level of methyltransferase activity in the cell. As used herein, the terms “determining,” “measuring,” and “assessing,” and “assaying” are used interchangeably and include both quantitative and qualitative determinations. In certain instances, the methyltransferase activity is assessed by detecting the production of 2′, 3′-cGAMP in the cell.

Cells of interest which may be targeted for assessing a methyltransferase activity and/or cGAMP levels in the subject methods include, but are not limited to, stem cells, T cells, dendritic cells, B Cells, granulocytes, leukemia cells, lymphoma cells, virus cells (e.g., HIV cells) NK cells, macrophages, monocytes, fibroblasts, epithelial cells, endothelial cells, and erythroid cells. Target cells of interest include cells that contain, or are suspected of containing a SAM-dependent methyltransferase. In some embodiments, the target cell is selected from a virus-infected cell, a regulatory T (Treg) cell, an antigen-specific T-cell, a tumor cell, or a hematopoietic progenitor cell (CD34⁺ cell). In certain embodiments, a cell extract of the target cells is utilized in the subject methods. In some cases of the subject methods, an extracted cell lysate of the target cells is utilized in the subject methods. In certain instances, a subject construct is transcribed in the cell before a cell extract is produced.

Any convenient 2′, 3′-cGAMP-producing nucleotidyltransferases may be targeted for assay according to the subject methods. Nucleotidyltransferases of interest include those enzymes that bind to microbial DNA as well as self DNA that invades the cytoplasm, and catalyzes cGAMP synthesis. In some embodiments, the nucleotidyltransferases is a cGAS. cGASs of interest include, but are not limited to, those described by Sun et al. (Cyclic GMP-AMP synthase is a cytosolic DNA sensor that activates the type I interferon pathway. Science 339 (6121): 786-91).

The subject methods may be performed using any convenient assay formats. In some cases, the method is performed using a flow cytometer. In some instances, the method further includes flow cytometrically analyzing the target cell. Detecting the cell in a flow cytometer may include exciting a biosensor with one or more lasers at an interrogation point of the flow cytometer, and subsequently detecting fluorescence emission from the signaling chromophore using one or more optical detectors. In some embodiments, the methods further include counting and/or sorting the target cells according to the biosensor fluorescence.

### Screening Assays

In some cases, the subject methods further comprises monitoring fluorescence of the signaling chromophore upon application of a stimulus to the cell. Any convenient stimuli may be applied to the cells depending on the application of interest, e.g., including research application involving the investigation of a cGAS of interest and drug discovery applications involving the identification of an agent that binds to a cGAS of interest. In some cases, application of a stimulus to the cell refers to contacting the cell with an agent of interest.

Aspects of the present disclosure also include screening assays configured to screen, identify and/or assess the activity of enzyme(s) that modulate or regulate cGAMP in a sample. In certain cases, the enzyme is a phosphodiesterase that cleaves 2′,3′-cGAMP. In certain cases, the enzyme is cGAS. Screening assays of interest include methods of assessing whether an enzyme of interest modulates the level of cGAMP in a sample. In some cases, the specific detection of 2′, 3′-cGAMP either produced or removed by the action of an enzyme of interest provides for a sensitive assay of the enzyme modulation activity. A subject biosensor may be introduced to a sample in vitro, and the behavior of an enzyme of interest in the sample may be assessed.

Aspects of the present disclosure also include screening assays configured to identify agents that find use in modulating the activity of a cGAS of interest, e.g., as reviewed above. Screening assays of interest include methods of assessing whether a test compound modulates the activity of a cGAS of interest. By assessing is meant at least predicting that a given test compound will have a desirable activity, such that further testing of the compound in additional assays, such as animal model and/or clinical assays, is desired. The specific detection of 2′, 3′-cGAMP produced by the action of cGAS provides for a sensitive assay of cGAS activity. A subject biosensor may be introduced to a sample in vitro, and the behavior of a cGAS of interest in the presence of the test compound may be assessed.

Drug screening may be performed using an in vitro model, a genetically altered cell or animal (e.g., non-human animal), or purified cGAS of interest. One can identify ligands or substrates that compete with, modulate or mimic the action of a 2′, 3′-cGAMP-producing cGAS. Drug screening can identify agents that modulate cGAS activity, either as an antagonist or as an agonist. A wide variety of assays may be adapted for use in conjunction with the subject methods, including labeled in vitro protein-protein binding assays, electrophoretic mobility shift assays, immunoassays for protein binding, and the like.

The term “agent” as used herein describes any molecule, e.g., protein, small module, or pharmaceutical, with the capability of modulating a cGAS of interest. In some cases, a plurality of assay mixtures are run in parallel with different agent concentrations to obtain a differential response to the various concentrations. In some cases one of these concentrations serves as a negative control, i.e., at zero concentration or below the level of detection.

Candidate agents encompass numerous chemical classes, such as organic molecules, e.g., small organic compounds having a molecular weight of more than 50 and less than about 2,500 daltons. Candidate agents may include functional groups necessary for structural interaction with a target, particularly hydrogen bonding, and in some cases include at least an amine, carbonyl, hydroxyl or carboxyl group, in some cases at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

### Kits

Aspects of the present disclosure include kits, where the kits include one or more components employed in the biosensors and methods of inventions, e.g., nucleic acids, constructs, host cell, as described herein. In some embodiments, the kit includes a single stranded nucleic acid or a nucleic acid construct encoding the single stranded nucleic acid (e.g., as described herein). The subject kit may further include one or more components selected from a DFHBI fluorophore, 2′, 3′-cGAMP, a promoter, a cell, a cloning vector and an expression cassette. Any of the components described herein may be provided in the subject kit, e.g., cells comprising biosensor systems, biosensors, nucleic acids, constructs (e.g., vectors) encoding for components of the biosensor, e.g., aptamer domains, genomic constructs, components suitable for use in expression systems (e.g., cells, cloning vectors, multiple cloning sites (MSC), bi-directional promoters, an internal ribosome entry site (IRES), etc.), etc. A variety of components suitable for use in making and using constructs, cloning vectors and expression systems may find use in the subject kits. Kits may also include tubes, buffers, etc., and instructions for use. The various reagent components of the kits may be present in separate containers, or some or all of them may be pre-combined into a reagent mixture in a single container, as desired.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another form of these instructions is a computer readable medium, e.g., diskette, compact disk (CD), Hard Drive etc., on which the information has been recorded. Yet another form of these instructions that may be present is a website address which may be used via the internet to access the information at a removed site.

### Utility

The biosensors, nucleic acids, constructs and methods of the present disclosure, e.g., as described herein, find use in a variety of different applications where it is desirable to detect 2′, 3′-cGAMP or investigate a cGAS/STING pathway member of interest. Applications of interest include, but are not limited to, research applications and diagnostic applications. The present disclosure also finds use in applications where biological samples may be assessed for cGAS activity for research, laboratory testing or for use in therapy.

The present disclosure provides a direct method of detecting and quantifying 2′, 3′-cGAMP rather than downstream signals, for diagnostic purposes and for clearly distinguishing cGAS/STING from other nucleic acids. The present disclosure provides detection methods for 2′, 3′-cGAMP that are adaptable to a high-throughput screening format, which can find use in biomedical applications, including but not limited to, development of small molecule modulators of the cGAS/STING pathway for cancer immunotherapy and vaccine development. The subject methods can find use in methods of screening and/or identifying enzymes that modulate cGAMP, such as an phosphodiesterase enzyme that cleaves 2′,3′-cGAMP. While current efforts have focused on targeting STING with natural and unnatural cyclic dinucleotides and the small molecule, cGAS can be an advantageous drug target because activating the enzyme can produce an amplified signal relative to the STING receptor-small molecule interaction. cGAS can also be an attractive target for small molecule inhibition in the case of autoimmune diseases. The present disclosure provides a direct and high-throughput method to assay cGAS activity which finds use in both fundamental and applied studies of the cGAS/STING immune signaling pathway.

The following examples are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Design of a Riboswitch-Based Fluorescent Biosensors for 2′,3′-cGAMP

Fluorescent biosensors were developed that exhibit turn-on response to 2′, 3′-cGAMP which find use in direct and high-throughput methods to assay cGAS activity and in the fundamental and applied studies of the cGAS/STING immune signaling pathway. The natural protein receptor for 2′, 3′-cGAMP, STING, is poorly suited for engineering a fluorescent biosensor, because it functions as a homodimer and its structure precludes facile connection of the protein chains or circular permutation. Overexpression of STING-based constructs in vivo also may activate downstream responses, which would be an undesired physiological effect. In contrast, the subject fluorescent biosensors exhibit turn-on response to 2′, 3′-cGAMP.

The biosensors were designed by making particular mutations to natural riboswitch aptamers of the GEMM-II class that recognize the related molecule 3′, 3′-cyclic di-GMP (c-di-GMP) (Lee et al., (2010). An allosteric self-splicing ribozyme triggered by a bacterial second messenger. Science 329, 845-848). Using one of these 2′, 3′-cGAMP biosensors, direct detection of cGAS enzymatic activity was demonstrated by fluorescence read-out in a 384-well format, fluorescence turn-on in flow cytometry upon overexpression of cGAS in live bacterial cells, and fluorescence-based quantitation of endogenous cGAS activity in DNA-stimulated lysates from the L929 mammalian cell line. In vitro enzymatic assays showed that nucleic acid intercalators can indirectly inhibit cGAS activity. The flow cytometry assays in bacteria demonstrated proof-of-principle for high-throughput, single cell measurement of intracellular 2′, 3′-cGAMP levels. Finally, analysis of mammalian cell lysates revealed that 60 attomoles, or 6 million molecules of 2′, 3′-cGAMP, are produced on average per cell upon DNA stimulation in the L929 cell line. These results demonstrate quantitation of cellular levels of 2′, 3′-cGAMP.

### Results

Engineering a Fluorescent Biosensor to Sense 2′, 3′-cGAMP

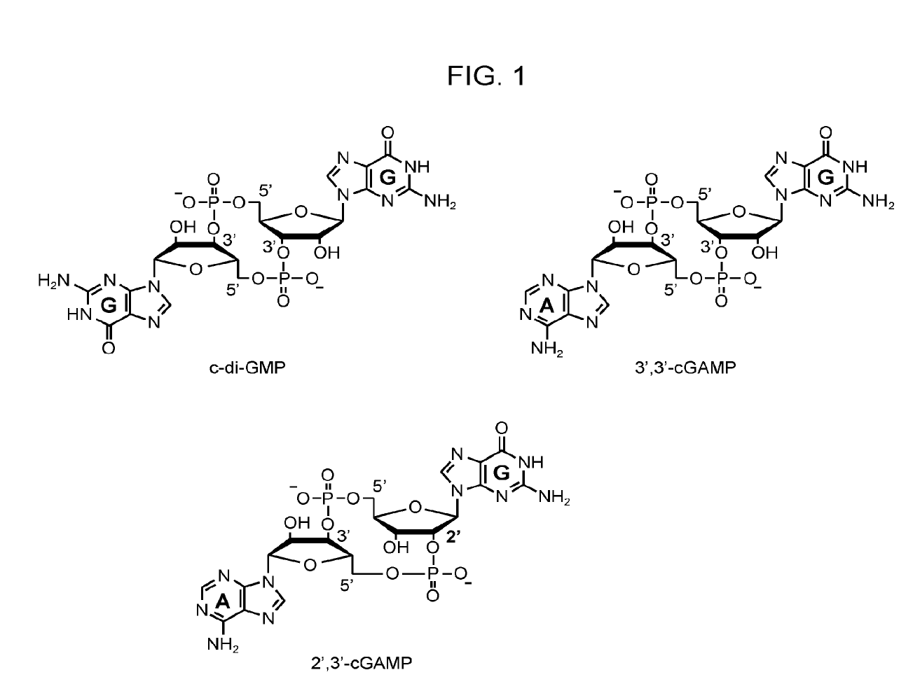

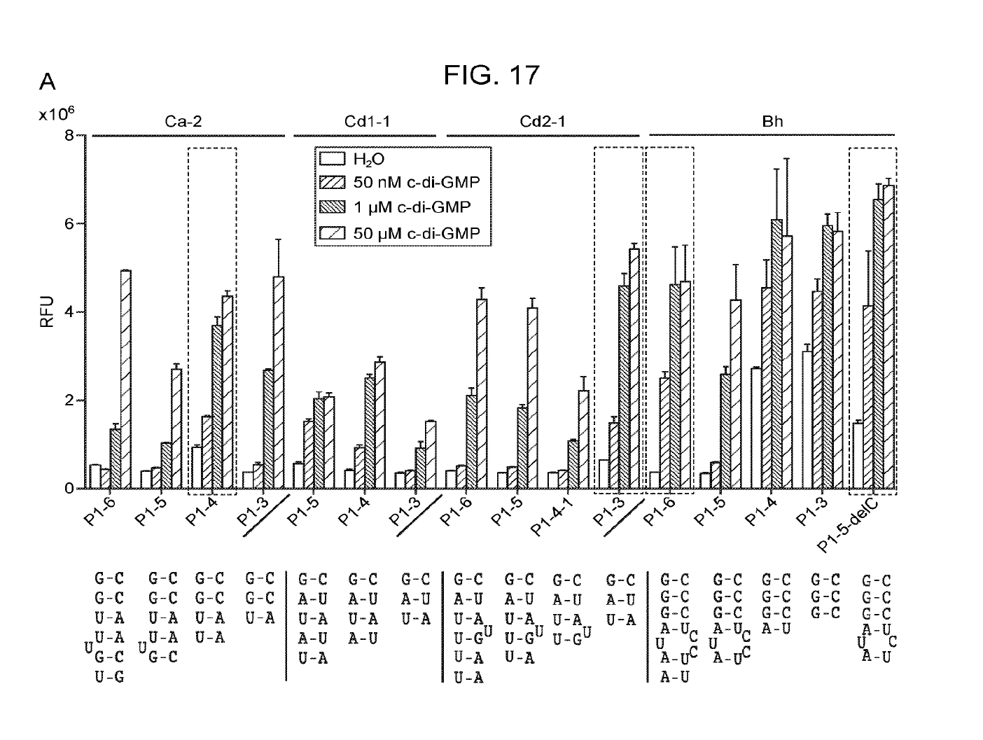

Previously, RNA-based fluorescent biosensors selective for c-di-GMP or 3′, 3′-cGAMP (FIG. 1A) were developed, based on natural GEMM-I riboswitches (Kellenberger et al., (2015). GEMM-I riboswitches from Geobacter sense the bacterial second messenger cyclic AMP-GMP. Proc Natl Acad Sci USA 112, 5383-5388; Kellenberger et al., RNA-based fluorescent biosensors for live cell imaging of second messengers cyclic di-GMP and cyclic AMP-GMP. J Am Chem Soc 135, 4906-490). However, these biosensors did not respond at all to 2′, 3′-cGAMP, even though some of the parent riboswitch aptamers exhibited very high specificity and affinity to 3′, 3′-cGAMP (see also Ren et al., (2015). Structural basis for molecular discrimination by a 3′,3′-cGAMP sensing riboswitch. Cell Rep 11, 1-12). X-ray crystal structures of ligand-bound GEMM-I riboswitches showed extensive interactions with the 2′-hydroxyls and phosphodiester oxygens of c-di-GMP or 3′, 3′-cGAMP (FIG. 16) (Ren et al., 2015; Smith et al., (2011). Structural basis of differential ligand recognition by two classes of bis-(3′-5′)-cyclic dimeric guanosine monophosphate-binding riboswitches. Proc Natl Acad Sci USA 108, 7757-7762). These interactions would be disrupted by the 2′-5′ linkage present in 2′, 3′-cGAMP.

The GEMM-II riboswitch scaffold was selected as being more amenable for engineering a 2′, 3′-cGAMP biosensor. The canonical ligand for this riboswitch class is c-di-GMP, however x-ray crystal structures showed that there are few canonical Watson-Crick or Hoogsteen pairing interactions between ligand nucleobases and the riboswitch (FIG. 16) and few hydrogen bonds between the ligand backbone and the riboswitch. This suggests that GEMM-II may have some flexibility in base recognition. Furthermore, the GEMM-II riboswitch aptamer from Clostridium acetobutylicum can accept c-di-GMP analogs with modifications to the ribose and phosphates (Shanahan et al., (2011). Differential analogue binding by two classes of c-di-GMP riboswitches. J Am Chem Soc 133, 15578-15592; Smith et al., 2011), which was promising for tolerating changes to the backbone linkage.

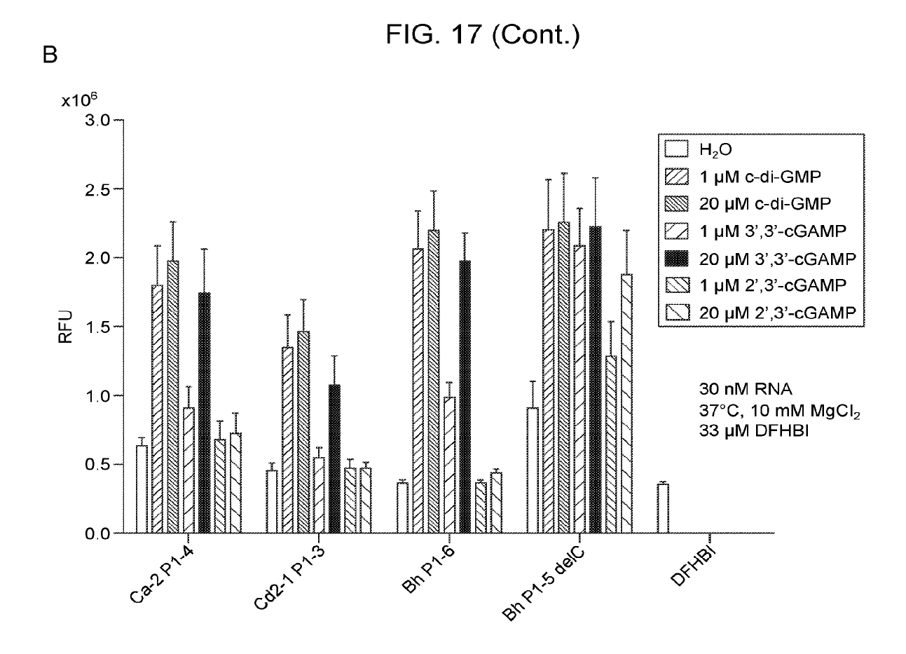

Four characterized GEMM-II riboswitches (Lee et al., (2010). An allosteric self-splicing ribozyme triggered by a bacterial second messenger. Science 329, 845-84; Smith et al., 2011) were picked to design 16 biosensor candidates with varied length transducer stems derived from the natural riboswitch P1 stems (Table 1). These biosensors were initially screened for fluorescence turn-on and binding affinity to c-di-GMP, the canonical ligand (FIG. 17, panel A), then the four most promising biosensors were further profiled for response to c-di-GMP, 3′, 3′-cGAMP, and 2′, 3-cGAMP (FIG. 17, panel B). One of the candidates that incorporated a GEMM-II riboswitch from Bacillus halodurans C-125, Bh P1-5 delC, exhibited fluorescence response to micromolar concentrations of 2′, 3′-cGAMP. The name indicates that the riboswitch portion of the biosensor is fused to the Spinach2 aptamer via a 5 base-paired P1 stem with deletion of a single C present in the natural stem bulge (FIG. 2).

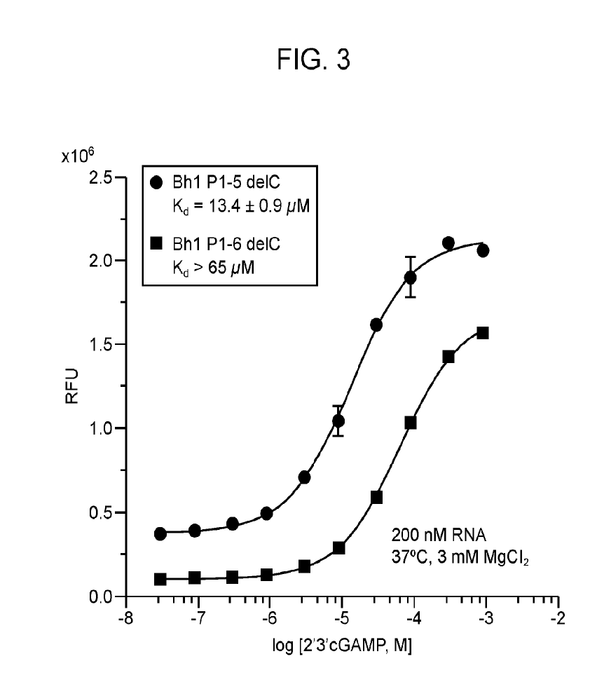

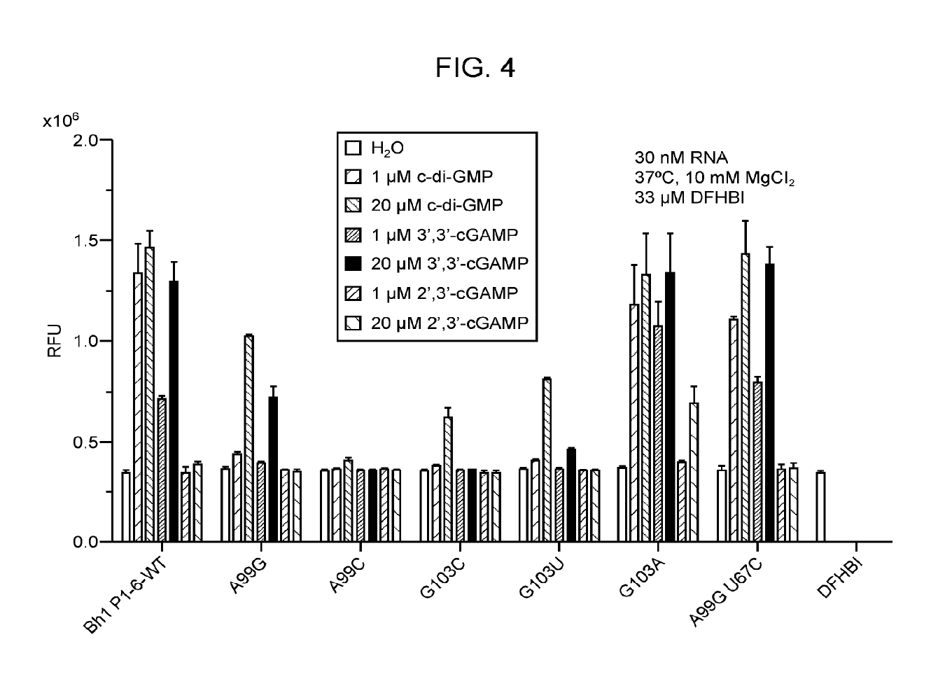

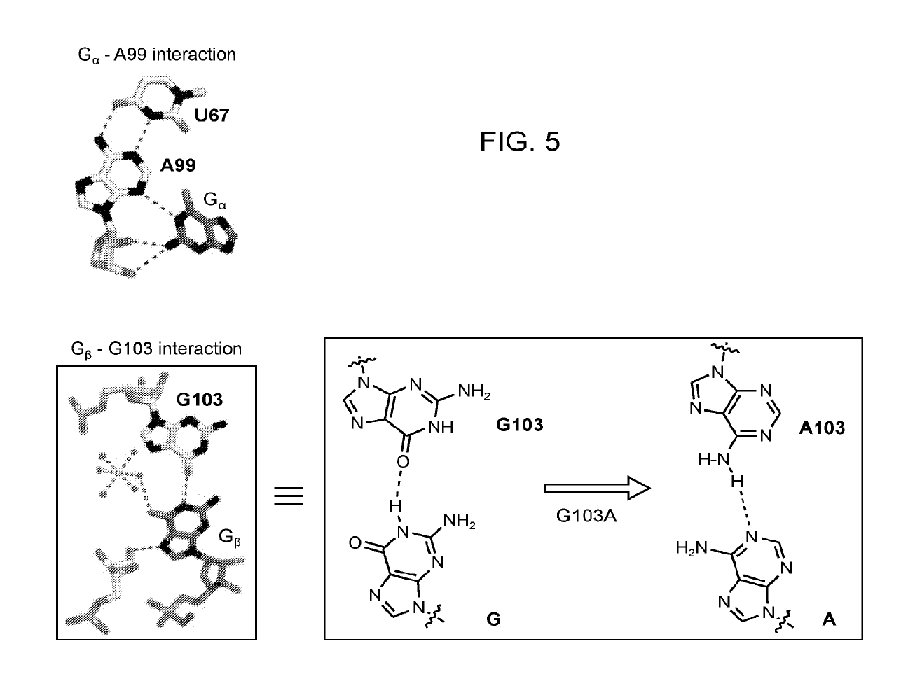

While Bh P1-5 delC showed good affinity for 2′, 3′-cGAMP (apparent dissociation constant or K_{D}=13.4±0.9 μM) at 37° C., 3 mM Mg²⁺ (FIG. 3), the related Bh P1-6 showed lower background fluorescence and higher activation (5-fold) with c-di-GMP (FIG. 17, panel B), which allowed us to analyze the effect of mutations on ligand selectivity. Two positions in the riboswitch binding pocket that make direct contacts with the ligand nucleobases were mutagenized and the fluorescence response to 2′, 3′-cGAMP was examined (FIGS. 4, 5). Whereas mutations to A99, the position that recognizes G_{α} of c-di-GMP, did not improve response to 2′, 3′-cGAMP, fluorescence activation by 2′, 3′-cGAMP with G103A, which mutates the position that recognizes G_{β} of c-di-GMP, was observed.

The effect of the G103A mutation may establish a hydrogen bond with the A base of 3′, 3′-cGAMP or 2′, 3′-cGAMP, and disfavor interaction with the G base of c-di-GMP. Accordingly, it was found that the cyclic dinucleotide selectivity profile for Bh P1-6 G103A biosensor was switched relative to wild-type Bh P1-6 (Table 2). Binding affinity to 3′, 3′-cGAMP was increased by more than 10-fold (K_{D} from >10 μM to 0.8 μM), whereas binding affinity to c-di-GMP was reduced by more than 125-fold (K_{D} from 56 nM to >7 μM). Furthermore, improved binding extended to 2′, 3′-cGAMP, such that 200 nM of Bh P1-6 G103A showed 5-fold fluorescence activation in response to 20 μM ligand, whereas the wild-type biosensor had shown no response (FIGS. 3 and 17, panel B).

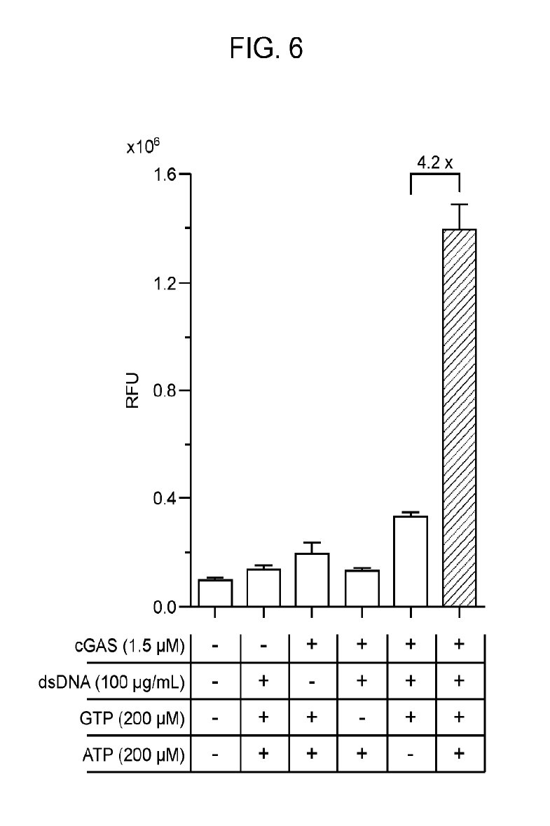

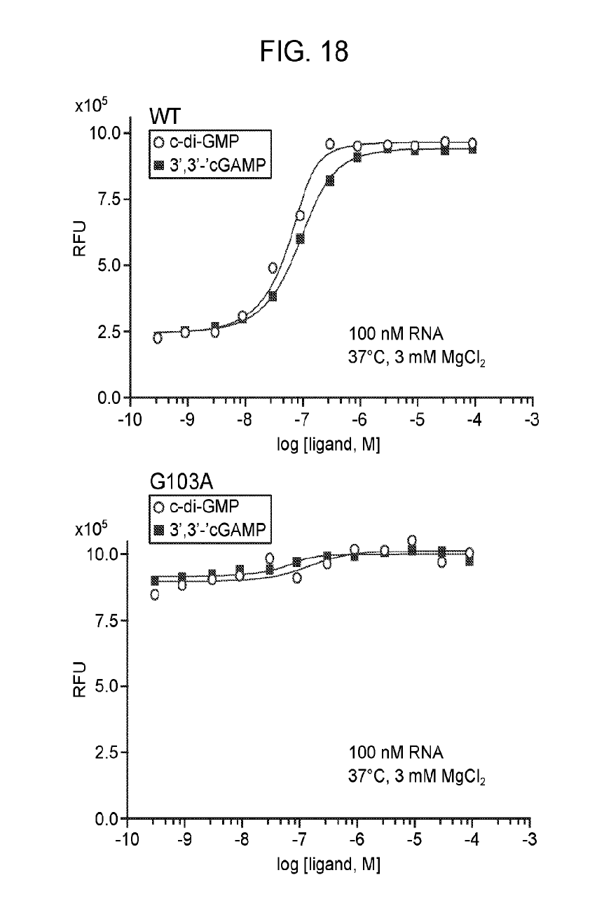

It was expected that the same G103A mutation could be applied to the Bh P1-5 delC biosensor, but disappointingly, it resulted in complete loss of biosensor function (FIG. 18). High fluorescence was observed even in the absence of ligand, which implies that this single nucleotide substitution, in conjunction with deleting one nucleotide in the stem bulge, favors formation of a stable transducer stem in the absence of ligand. Nevertheless, by combining rational design and structure-based mutagenesis, two fluorescent biosensors have been developed capable of detecting 2′, 3′-cGAMP at micromolar concentrations. A detailed comparison of their binding affinities and fluorescent turn-on properties is given in Table 2. Similar to STING, the native protein receptor for 2′, 3′-cGAMP, the biosensors also respond to the bacterial second messengers c-di-GMP and 3′, 3′-cGAMP, but otherwise exhibit high selectivity against other metabolites such as ATP and GTP (FIG. 6). Importantly, whereas STING is a homodimeric protein that is not readily adaptable as a fluorescent biosensor, the present disclosure provides a first-generation fluorescent biosensor for 2′, 3′-cGAMP based on the GEMM-II riboswitch scaffold.

Assaying cGAS Activity In Vitro Using a Fluorescent Biosensor

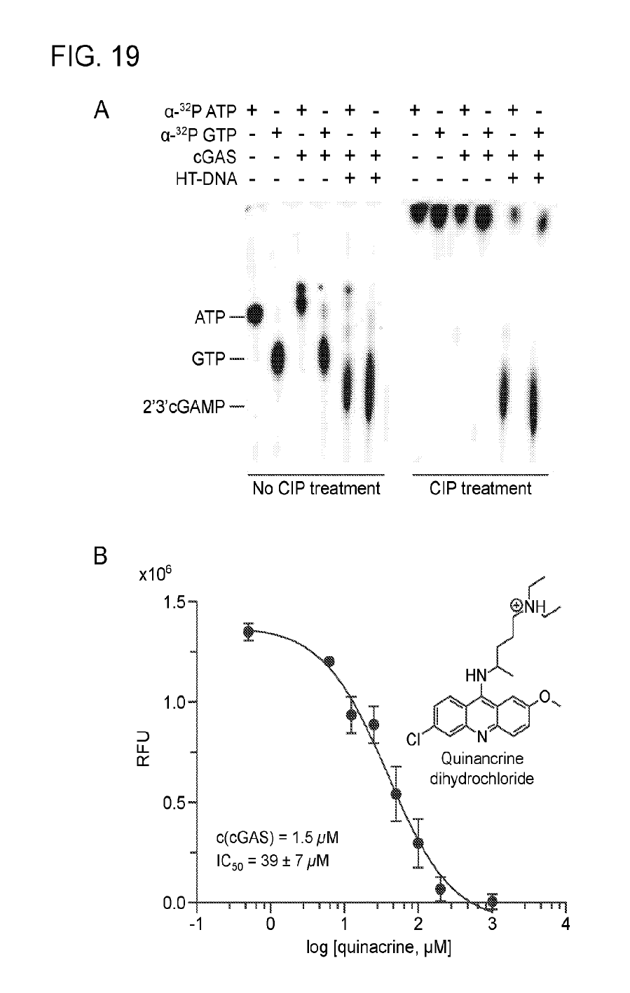

Currently, one established method to assay cGAS activity in vitro is via thin layer chromatography analysis of radiolabeled nucleotides (FIG. 19) (Diner et al., (2013). The innate immune DNA sensor cGAS produces a noncanonical cyclic dinucleotide that activates human STING. Cell Rep 3, 1355-136).

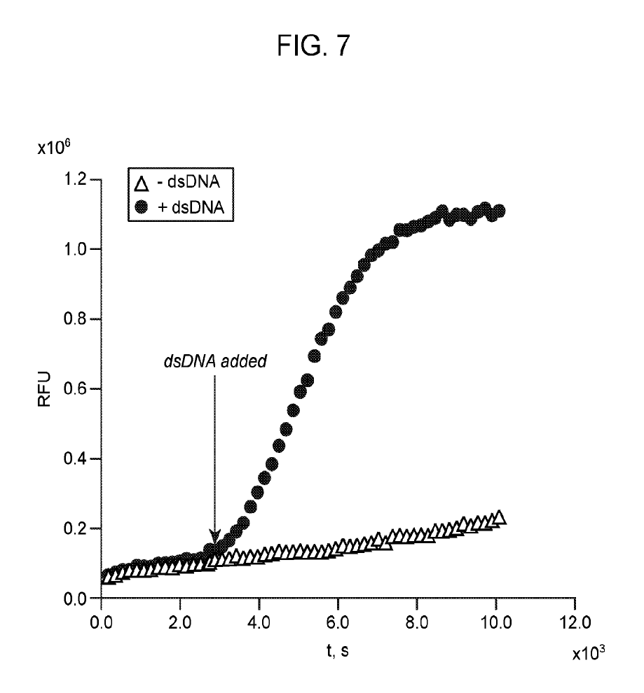

Enzymatic activity of the DNA sensor cGAS was analyzed using the Bh P1-6 G73A biosensor, which has lower background fluorescence and higher turn-on. Upon binding to double-stranded DNA (dsDNA), cGAS is activated to catalyze the 2′-5′ phosphodiester bond formation between GTP and ATP, followed by cyclization to form the 3′-5′ bond (Ablasser et al., (2013). cGAS produces a 2′-5′-linked cyclic dinucleotide second messenger that activates STING. Nature 498, 380-384). As expected, the biosensor gives strong fluorescence signal only when all components of the reaction are present to provide robust production of 2′, 3′-cGAMP (FIG. 6). Besides carrying out assays in a standard mix-and-go format, in which the enzyme reactions are performed separately from the biosensor detection reactions, it was also showed that the biosensor can be employed for direct in situ detection, e.g. the biosensor functions in the enzymatic reaction buffer (FIG. 7).

Minor fluorescence increases were also detected in control reactions without dsDNA or without ATP. The former due to incomplete removal of dsDNA during purification of cGAS enzyme, resulting in the observed basal activation. The latter could be caused by the formation of either pppGp(2′-5′)G or 2′, 3′-c-di-GMP, which have been previously reported as minor products in the absence of ATP (Ablasser et al., 2013). Biosensor response to these side products was not tested because of the difficulty in obtaining sufficient amounts for binding measurements.

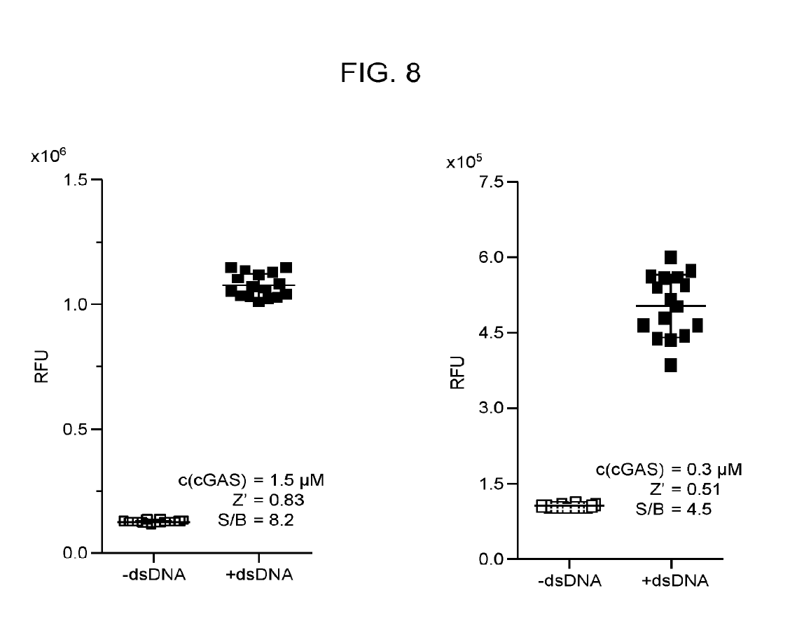

Since the biosensor-based fluorescent assay can be performed in a 384-well plate and analyzed in a fluorescent plate reader, it is readily adapted to high-throughput screening for activators or inhibitors of cGAS. With 1.5 μM concentration of cGAS, a Z′ score of 0.83 was determined for the assay in this format, where a Z′ score >0.5 is considered an assay with excellent statistical reliability for HTS (FIG. 8) (Zhang et al., (1999). A Simple Statistical Parameter for Use in Evaluation and Validation of High Throughput Screening Assays. J Biomol Screen 4, 67-73). In order to screen for high affinity inhibitor or activator compounds, the assay would need to be performed with nanomolar concentrations of enzyme. A Z′ score of 0.51 was obtained using the RNA biosensor, 300 nM enzyme, and manual pipetting of all reagents in 384-well format (FIG. 8). Thus, the current fluorescent biosensor provides HTS assays for moderate affinity (e.g. 300-500 nM dissociation constant) compounds that affect cGAS activity.

Determining Mechanism of Small Molecule Inhibition of cGAS

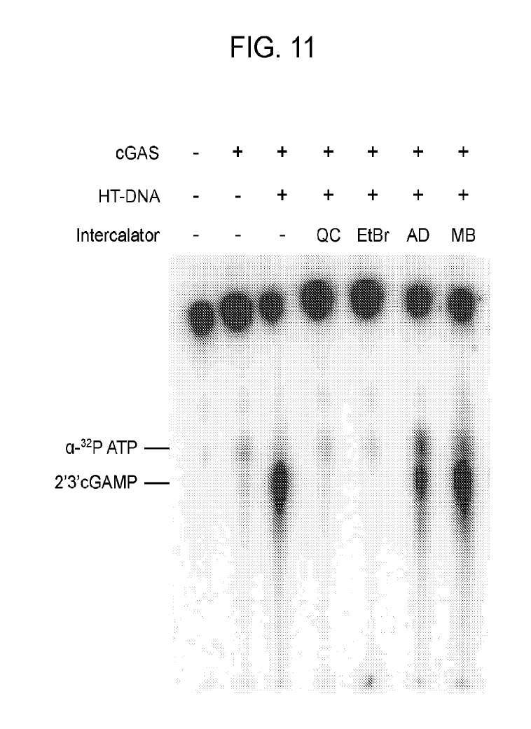

Quinacrine, a well-characterized anti-malarial drug, has been reported to inhibit cGAS activity, with an IC₅₀ value of 13 μM measured by the radiolabeled TLC assay (Gray et al., (2015). Cutting Edge: cGAS Is Required for Lethal Autoimmune Disease in the Trex1-Deficient Mouse Model of Aicardi-Goutieres Syndrome. J Immunol 195, 1939-1943). Using the fluorescent biosensor assay, an apparent IC₅₀ of 39±7 μM (FIG. 3A) was measured, but a control experiment revealed that the decrease in fluorescence signal was independent of enzyme activity (FIG. 3B). Quinacrine is a DNA intercalator that may disrupt biosensor function by intercalating into the RNA helices. It was shown that quinacrine also decreased fluorescence of the dye-binding Spinach2 aptamer (FIG. 10). Furthermore, the compound likely inhibits cGAS activity indirectly, by interfering with DNA binding.

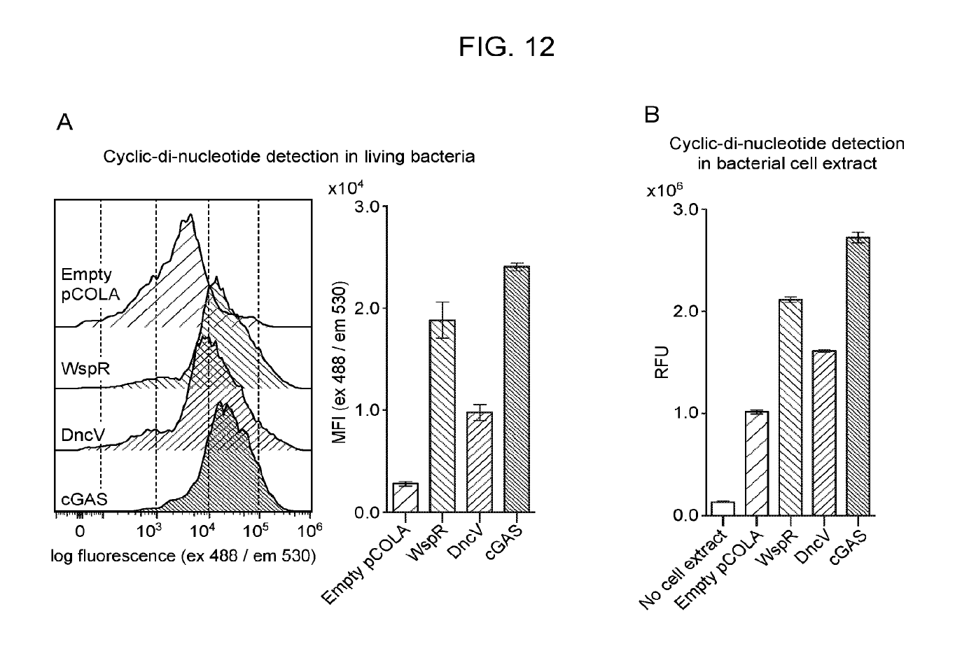

Quinacrine intercalation can lengthen the B-form DNA helix but otherwise would not grossly alter conformation (Hossain et al., (2008). DNA intercalation by quinacrine and methylene blue: a comparative binding and thermodynamic characterization study. DNA Cell Biol 27, 81-90). To support the proposed mechanism of action, other DNA intercalators were tested for effects on cGAS activity. The radiolabeled TLC assay was employed in order to avoid possible complications in data interpretation from compounds interacting with the RNA biosensor. Quinacrine and ethidium bromide have similar DNA binding modes (LePecq and Paoletti, (1967). A fluorescent complex between ethidium bromide and nucleic acids. Physical-chemical characterization. J Mol Biol 27, 87-106), and both fully inhibited cGAS activity at 100 μM concentration. Actinomycin D, which binds to ssDNA and dsDNA, showed partial inhibition at that concentration, whereas methylene blue, which intercalates in a completely different orientation than quinacrine (Hossain et al., 2008), had no effect (FIG. 11). These results provide strong evidence that nucleic acid intercalators are a class of small molecule compounds capable of inhibiting cGAS, albeit via an indirect mechanism, shifting the helix conformation of the DNA ligand. Importantly, the enzyme-independent effect of compounds on fluorescence of the RNA biosensors can easily be analyzed in control reactions, which allows one to distinguish compounds in the screen that likely interact with the DNA instead of the enzyme directly.

Measuring Activity of Expressed cGAS in Live Bacterial Cells

The performance of the 2′, 3′-cGAMP riboswitch-based biosensor under in vivo conditions was assessed. The biosensor construct was inserted into a tRNA scaffold (Kellenberger et al., (2015) Live Cell Imaging Using Riboswitch-Spinach tRNA Fusions as Metabolite-Sensing Fluorescent Biosensors. Methods Mol Biol 1316, 87-103; Ponchon and Dardel, (2007). Recombinant RNA technology: the tRNA scaffold. Nat Methods 4, 571-576), then the fluorescent biosensor was co-expressed along with empty vector or enzymes that synthesize different cyclic dinucleotides in E. coli BL21-Star cells. WspR from Pseudomonas fluorescens produces c-di-GMP, DncV from Vibrio cholerae produces 3′, 3′-cGAMP, and cGAS from Homo sapiens produces 2′,3′-cGAMP. After incubating cells with the profluorescent dye DFHBI, the distribution of cellular fluorescence was analyzed by flow cytometry, which provides high-throughput, single-cell analysis of metabolites as detected by the fluorescent biosensor.

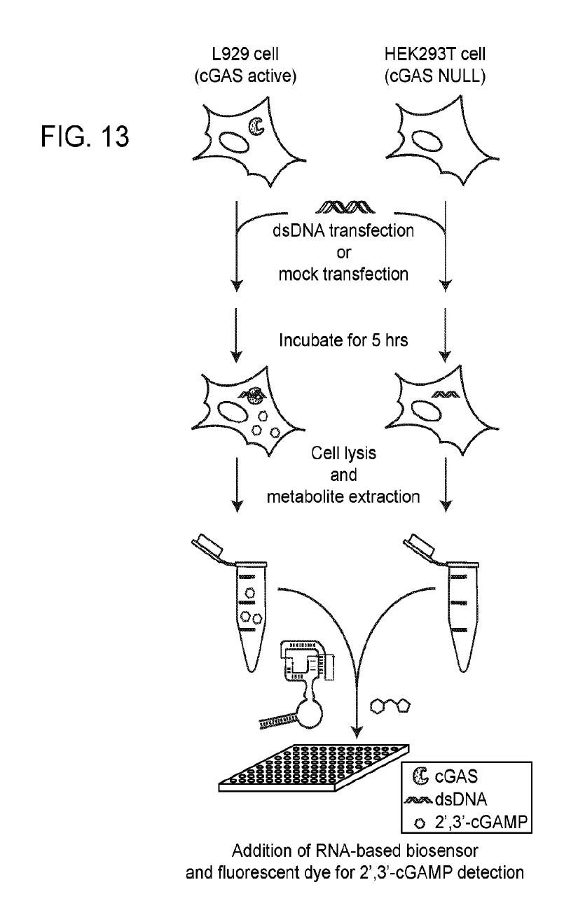

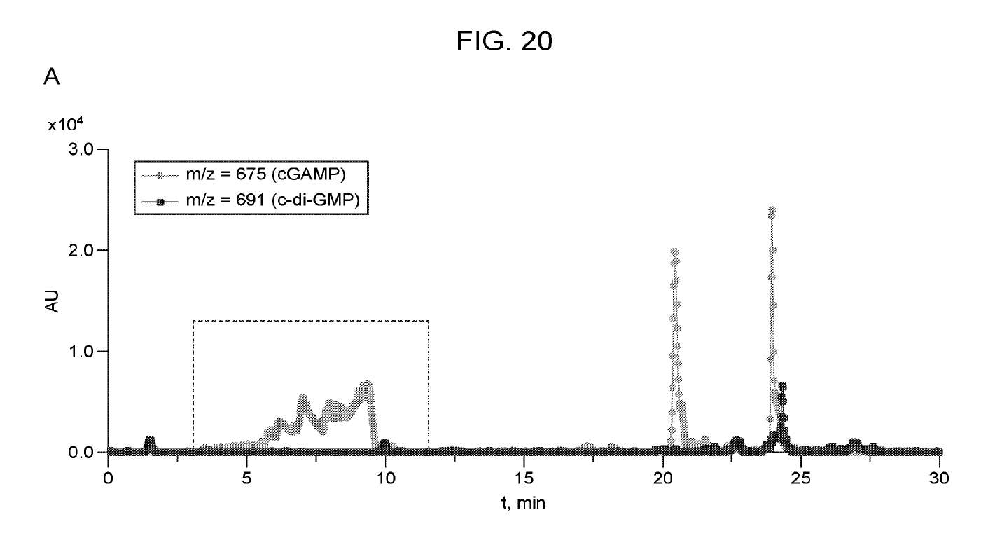

Significant fluorescence activation was observed upon co-expression of each enzyme (FIG. 12, panel A), because the Bh P1-6 G73A biosensor is capable of binding 3′, 3′-cGAMP, c-di-GMP, and 2′, 3′-cGAMP (listed in order of affinity). However, the degree of fluorescence activation did not correspond to the relative affinity of the bio sensors for the different cyclic dinucleotides. Instead, it appears to correlate directly to the amount of each cyclic dinucleotide produced by the enzymes in vivo, as the identical trend was observed when an aqueous-organic extraction of cell lysates was performed and the extracts containing cyclic dinucleotides were analyzed using the biosensor (FIG. 12, panel B). Notably, given that the biosensor has poorer affinity for 2′,3′-cGAMP versus the two 3′,3′-linked cyclic dinucleotides, this result implies that cGAS is highly activated by binding to endogenous genomic DNA to produce an extremely large amount of 2′, 3′-cGAMP in E. coli. In addition, there is likely no endogenous phosphodiesterase capable of efficiently hydrolyzing the compound. Accordingly, LCMS analysis showed that 2′, 3′-cGAMP is highly abundant in the cell extracts, building up to ˜0.5 mM concentration based on a standard curve (FIG. 20). It should also be noted that E. coli has endogenous c-di-GMP (Weber et al., (2006). Cyclic-di-GMP-mediated signalling within the sigma network of Escherichia coli. Mol Microbiol 62, 1014-1034), which explains why the biosensor is activated by extracts from cells harboring the empty pCOLA vector. Thus, the fluorescence activation of the biosensor observed in vivo is likely lower than the theoretical maximum fold activation, due to basal activation by endogenous c-di-GMP. However, this issue is mainly confined to analysis in bacteria, because mammalian cells generally do not produce c-di-GMP, except for some cells engineered for synthetic biology applications. In all, these live-cell flow cytometry results demonstrate that the subject first-generation biosensors are capable of detecting 2′,3′-cGAMP in the complex setting of bacterial cells, and can be utilized to achieve 2′,3′-cGAMP detection in live mammalian cells.

Quantitating 2′, 3′-cGAMP in Lysates of DNA-Stimulated Mammalian Cells

While the above results demonstrate that the fluorescent biosensor can detect cGAS activity in vitro and upon heterologous expression in the presence of DNA, a further aim is to detect cGAS activation in mammalian cells. 2′,3′-cGAMP is produced by cGAS+ mammalian cells as part of a cytosolic immune surveillance pathway for foreign DNA, which could be caused by viral or bacterial infection, and alternatively, by leakage of damaged nuclear or mitochondrial DNA. A standard method to simulate these conditions is to transfect cells with double-stranded DNA, however it has not been possible to directly measure the levels of 2′, 3′-cGAMP produced. Instead, the main method to study this pathway involves an IFN-β reporter assay, which requires prior transfection of the reporter DNA and is not specific to the cGAS-STING pathway, as other immune signaling also stimulate interferon production and activate the promoter controlling reporter expression. Furthermore, liquid chromatography-mass spectrometry (LC-MS) analysis is not high-throughput and is hampered by low sensitivity due to the fact that cyclic-di-nucleotides are negatively charged and ionize poorly, with a lower limit of detection of 5 μM in pure samples. 2′,3′-cGAMP by LC-MS was not detected in the cell lysate samples described below, and ion suppression significantly reduces the sensitivity of detection in mammalian cell lysates even further.

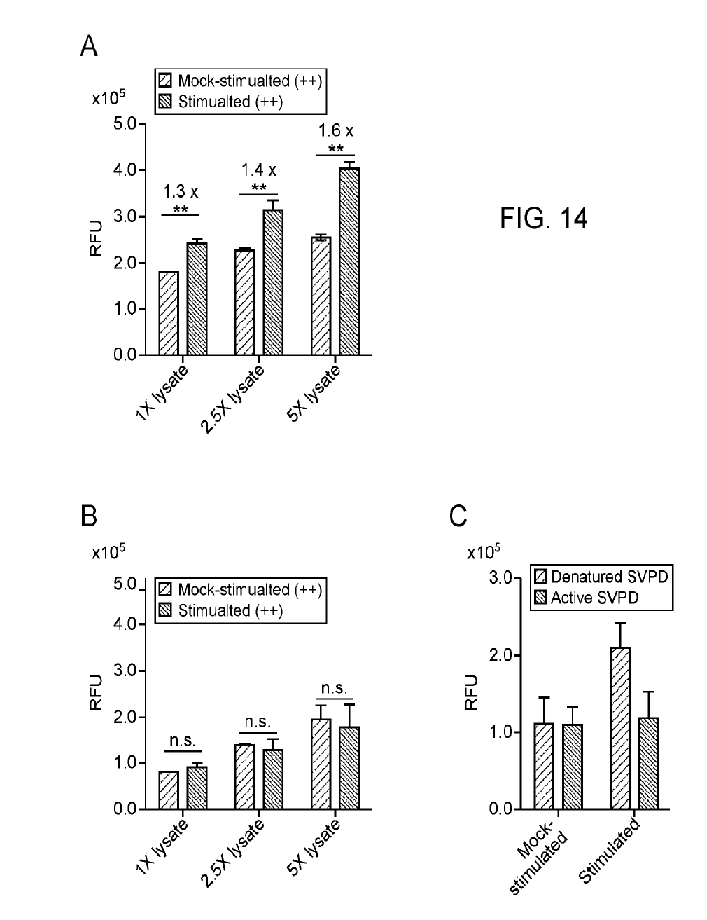

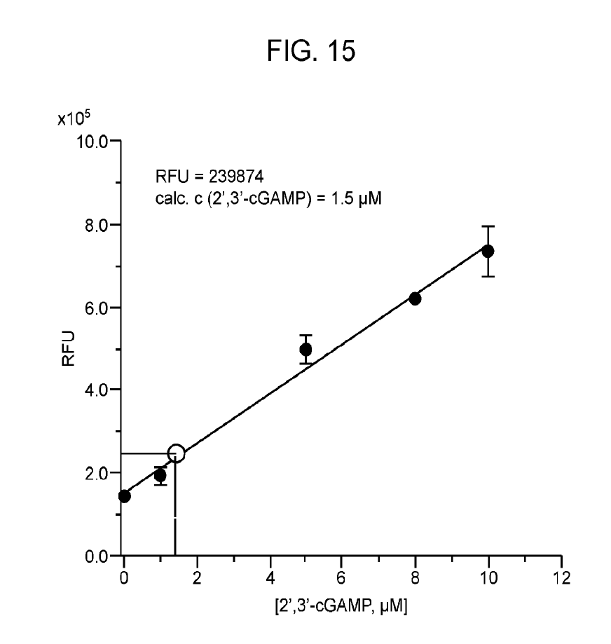

The ability of the fluorescent biosensor to detect 2′, 3′-cGAMP produced upon dsDNA stimulation of cGAS+L929 cells was evaluated. In comparison, mock-transfected L929 cells and HEK293T cells, which are cGAS null, were also analyzed under identical conditions. In vitro measurements were performed by adding aliquots of cell lysate to the fluorescent biosensor reaction in a 96-well plate reader (FIG. 13). Fluorescence activation was observed for dsDNA-stimulated over mock-stimulated L929 cells, with increasing turn-on signal upon addition of 1 to 5 μL of concentrated cell lysate (3×10⁵ cells per μL of concentrated cell extract) (FIG. 14, panel A). In contrast, no significant fluorescence change between dsDNA-stimulated and mock-stimulated was observed for HEK293T cells (FIG. 14, panel B). Thus, it was shown that the first-generation biosensor has sufficient sensitivity to detect endogenous 2′, 3′-cGAMP produced in response to dsDNA activation of cGAS enzyme in human cell lysates.

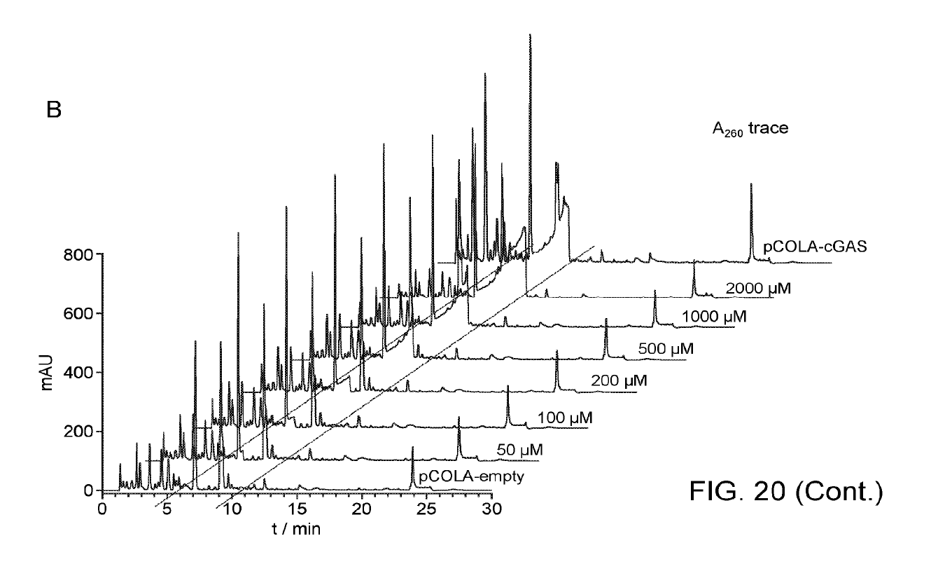

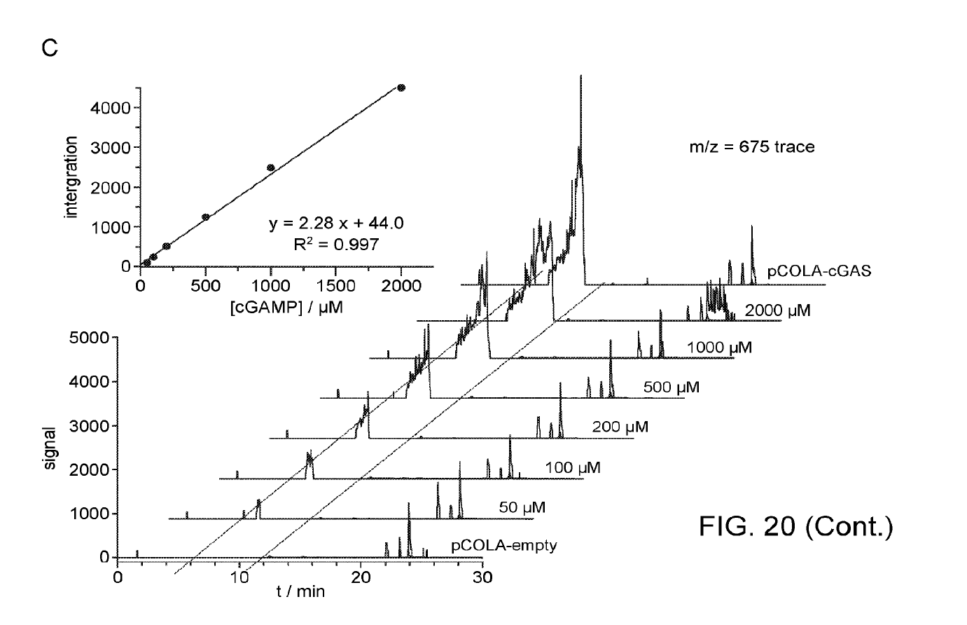

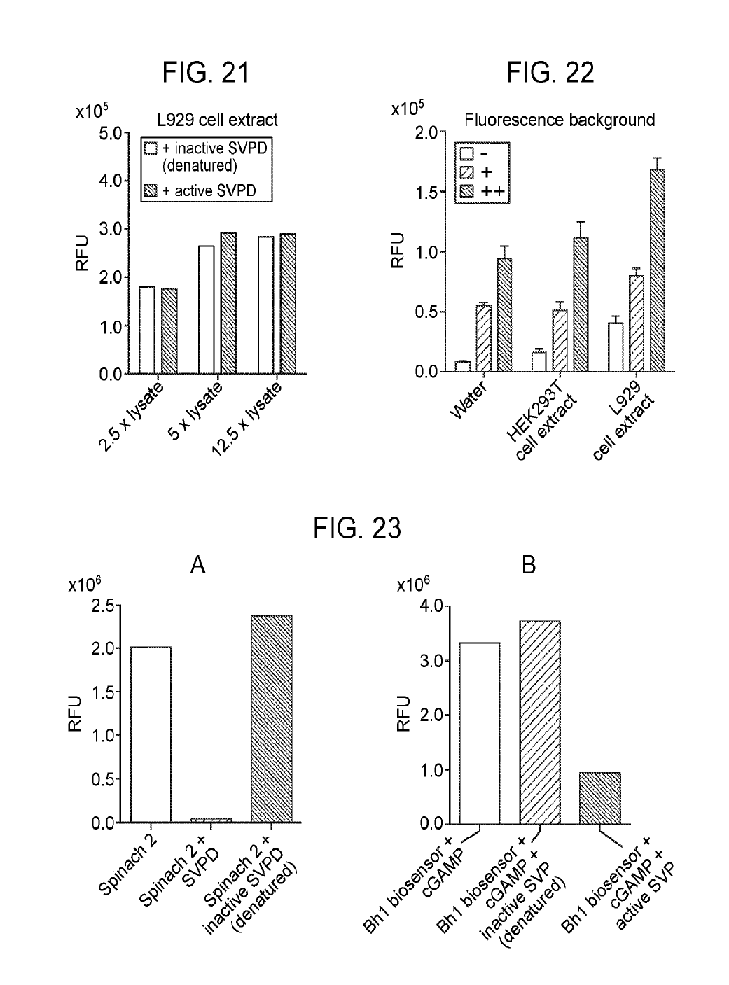

Interestingly, higher background fluorescence signal was observed for mock-stimulated L929 cells than HEK293T cells. It was checked whether there was basal 2′, 3′-cGAMP in mock-stimulated L929 cells that was activating the fluorescent biosensor. Background fluorescence for the mock-stimulated sample did not change upon treatment with active snake venom phosphodiesterase (SVPD), which degrades 2′, 3′-cGAMP (FIG. 14, panel C). In contrast, active SVPD did reduce the fluorescence signal of dsDNA-stimulated L929 cell lysates to the same levels as mock-stimulated samples. Therefore, the observed difference in background fluorescence was due to distinct levels of auto-fluorescence between the cell types and includes contributions to background fluorescence from the presence of the dye DFHBI and the RNA biosensor (FIGS. 21-23).

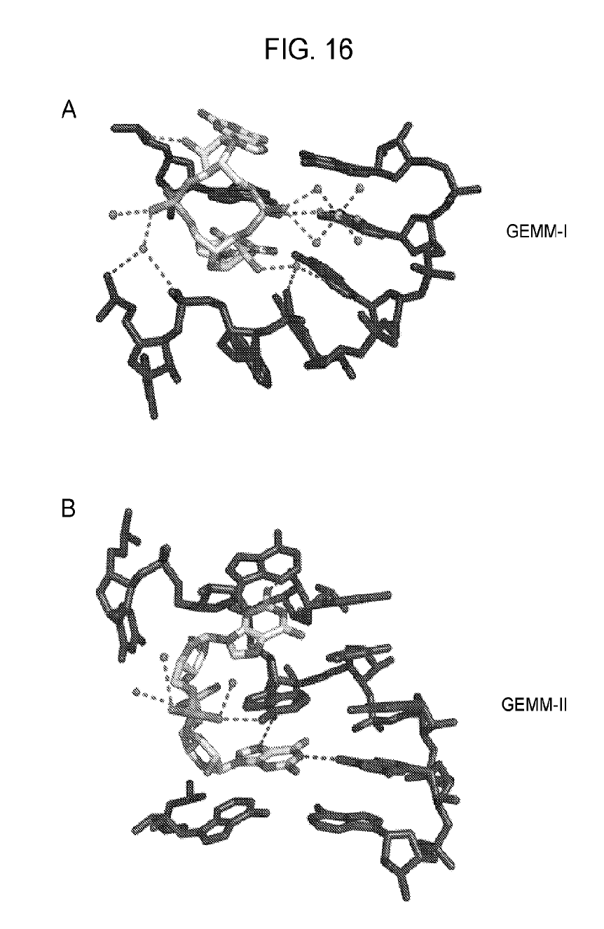

Finally, fluorescent biosensor was used to determine the absolute concentration of 2′, 3′-cGAMP produced upon dsDNA stimulation in L929 cells. Mock-stimulated L929 cell lysate was doped with known concentrations of 2′, 3′-cGAMP and the corresponding biosensor fluorescence signals were measured to obtain a standard curve with a lower limit of detection of 0.95 μM (FIG. 15). The fluorescence signal from L929 cells stimulated with dsDNA for 5 hours was compared to the standard curve, and showed that there was a concentration of 1.5 μM for 2′, 3′-cGAMP in concentrated L929 cell lysate. Assuming 100% extraction efficiency, it was calculated that DNA stimulation by transfection resulted in 60 attomoles of 2′, 3′-cGAMP produced on average per L929 cell, or 6 million molecules of 2′, 3′-cGAMP per cell (FIG. 15). This study provides exact measurements of endogenous 2′, 3′-cGAMP levels in mammalian cells.

### Discussion

No natural riboswitches for 2′, 3′-cGAMP have been discovered. RNA-based fluorescent biosensors for 2′, 3′-cGAMP were engineered via structure-based design. Initially, the GEMM-II scaffold was selected as a ligand-bound riboswitch structure of interest and particular mutations were designed into the ligand binding pocket. A screen of different phylogenetic and stem variants was designed that starts with the natural P1 stem. The results in this study demonstrate that riboswitch-based biosensors can be reprogrammed to recognize other ligands via point mutations. This indicates that natural riboswitch-ligand pairs can serve as starting points for the engineering of biosensors for related ligands not recognized by known riboswitches.

The subject biosensors exhibit fluorescence turn-on activity in direct response to 2′, 3′-cGAMP and thus are readily adapted for high-throughput screening assays of cGAS enzyme activity and inhibition. Given the emerging clinical relevance of the cGAS/STING innate immune signaling pathway for infectious diseases, cancer immunotherapy, and DNA-triggered autoimmune disorders, a fluorescent platereader screen for small molecule modulators of cGAS activity is a timely and valuable research tool. Furthermore, it was showed that the subject assay provides a straightforward way to distinguish between compounds that target cGAS enzyme versus interact with the activating ligand, double-stranded DNA.

It was found that intercalating compounds affect cGAS activation. This finding has broad implications for the design of synthetically modified double-stranded nucleic acids for therapeutic or research purposes. In particular, backbone modifications that favor B-form-like helical conformations may cause the nucleic acid agent to bind and activate cGAS, thus triggering interferon response. This result provides knowledge about the length and sequence requirements for cGAS activation, and further indicates that some synthetic modifications may be better at eluding cGAS surveillance and thus the cytosolic immune response. The subject assay provides a rapid, non-radioactive, and low material cost method to screen nucleic acid agents for such activity.

Finally, a fluorescent bio sensor was employed to quantitate the levels of 2′, 3′-cGAMP in L929 cell lysates, which previously had been shown to be cGAS+ via LC-MS analysis (Sun et al. (2013). Cyclic GMP-AMP synthase is a cytosolic DNA sensor that activates the type I interferon pathway. Science 339, 786-791), but not in a quantitative manner. An LC-MS/MS method has been developed for quantitation of 2′, 3′-cGAMP (Paijo et al., 2016, cGAS Senses Human Cytomegalovirus and Induces Type I Interferon Responses in Human Monocyte-Derived Cells. PLoS Pathog 12, e1005546). The amount of 2′, 3′-cGAMP produced upon HCMV virus infection was measured in the range of 0.5 to 3 attomoles on average per cell, depending on the cell type (Paijo et al., 2016). It was found that transfection of DNA leads to a much stronger response, as 60 attomoles of 2′, 3′-cGAMP were measured on average per cell at an earlier time point (6 h post-transfection versus 24 h post-infection). Based on the standard curve in L929 lysates, the current lower limit of detection of an exemplary fluorescent biosensor is ˜1 μM or 40 attomoles per cell. Thus, improving biosensor sensitivity by 10- to 100-fold can provide a high-throughput method to analyze viral-induced cGAMP levels. This analysis indicates that second-generation fluorescent biosensors with the requisite higher sensitivity, e.g. binding affinities in the 10 to 100 nM range, can be useful for single-cell analysis of cGAMP production upon virus infection.

Much in the way that antibody-based assays are indispensable for analysis of kinase signaling, presented herein are fluorescent biosensor-based assays as a resource for the analysis of cyclic dinucleotide signaling. Measuring 2′, 3′-cGAMP levels is important for investigating the basic biology of the cGAS-STING signaling pathway and its mechanistic contribution to innate immune responses. A recent study showed that in a viral infection model, cGAS and STING expression levels do not necessarily correlate with 2′, 3′-cGAMP production, and furthermore 2′, 3′-cGAMP levels do not necessarily correlate with IFN production (Paijo et al., 2016). These findings underscore the importance of measuring cGAS enzyme activity, rather than using its expression level as a proxy. In the context of cancer immunology, it has been suggested that tumor-derived DNA activates cGAS activity in host immune cells (Woo et al., 2015, The STING pathway and the T cell-inflamed tumor microenvironment. Trends Immunol 36, 250-256), but direct proof of this hypothesis is still lacking. Addressing these and other questions about cGAS activity is facilitated by a high-throughput fluorescent platereader assay capable of analyzing 2′, 3′-cGAMP levels of different cell types under different conditions and at different time points in multiple replicates.

### Example 2: Materials and Methods

### Reagents and Oligonucleotides

DNA oligonucleotides for biosensor constructs were purchased as Ultramers from Integrated DNA Technologies (Coralville, Iowa) and other DNA oligonucleotides were purchased from Elim Biopharmaceuticals (Hayward, Calif.). DFHBI and DFHBI-1T were either purchased from Lucerna (New York, N.Y.) or were synthesized following previously described protocols and were stored as a 10-30 mM stock in DMSO. C-di-GMP, 3′, 3′-cGAMP, 2′, 3′-cGAMP were purchased from Axxora (Farmingdale, N.Y.). Commercially available reagents were used without further purification. T7 RNA polymerase, Phusion DNA polymerase were purchased from New England Biolabs Inc (Ipswich, Mass.). Chemically competent BL21 (DE3) Star cells were purchased from Life Technologies (Carlsbad, Calif.). cGAS inhibitor (Quinacrine dihydrochloride), HT-DNA, Snake venom phosphodiesterase (SVPD) was purchased from Sigma-Aldrich (St Louis, Mo.). L929 and HEK293T cells were purchased from ATCC (Manassas, Va.).

### In Vitro Transcription

DNA templates for in vitro transcription were prepared through PCR amplification using Phusion DNA polymerase (NEB) from sequence-confirmed plasmids using primers that added the T7 polymerase promoter sequence. PCR products were purified by QIAquick PCR purification kit (Qiagen) for characterization and application experiments. Templates were then transcribed using T7 RNA polymerase in 40 mM Tris-HCl, pH 8.0, 6 mM MgCl₂, 2 mM spermidine, and 10 mM DTT. RNAs were either purified by a 96-well format ZR-96 Clean & Concentrator (Zymo Research) or by denaturing (7.5 M urea) 6% PAGE. RNAs purified from PAGE were subsequently extracted from gel pieces using Crush Soak buffer (10 mM Tris-HCl, pH 7.5, 200 mM NaCl and 1 mM EDTA, pH 8.0). RNAs were precipitated with ethanol, dried, and then resuspended in TE buffer (10 mM Tris-HCl, pH 8.0, 1 mM EDTA). Accurate measurement of RNA concentration was determined by measuring the absorbance at 260 nm after performing a hydrolysis assay to eliminate the hypochromic effect due to secondary structure in these RNAs.

Expression and Purification of cGAS

Full length cGAS encoding plasmid was taken from Doudna lab (Kranzusch et. al. Cell report 2013). The protein was expressed at 16° C. in BL21-RIL DE3 E. coli (along with pRARE2 human tRNA plasmid (Agilent) grown in LB media for 20 hr after induction with 0.5 M IPTG. Cells were lysed by sonication in 20 mM HEPES (pH 7.5), 400 mM NaCl, 10% glycerol, 30 mM Imidazole, 1 mM PMSF, 1 mM TCEP. The lysate was treated with Dnase for 30 mins to remove any residual cGAS bound DNA. Clarified lysate was bound to Ni-NTA agarose (QIAGEN) and was washed with lysis buffer supplemented with 1 M NaCl. The bound protein was eluted using lysis buffer supplemented with 300 mM imidazole. The eluted protein was dialyzed overnight at 4° C. against buffer having 20 mM HEPES (pH 7.5), 150 mM KCl, 10% glycerol and 1 mM TCEP. While checking in sodium dodecyl sulfate-polyacrylamide gel electrophoresis (SDS PAGE), an additional protein, which co eluted with MBP tagged cGAS, was found. As the overall cGAS protein preparation was active, as checked by TLC based assay, the cGAS protein preparation was used in future assays. According to the Gel, it was assumed around 60% of the preparation product is the actual cGAS and the cGAS concentration was calculated accordingly.

Thin-Layer Chromatography Based cGAS Activity Assay

DNA-dependent human cGAS activity was assayed with recombinant full-length cGAS and HT-DNA (Sigma-Aldrich, St Louis, Mo.). cGAS (estimated final concentration 1.5 μM) or equal volumes of water were incubated with HT-DNA (final concentration 0.1 mg/ml) in the presence of 250 μM ATP and GTP and [a-32P] ATP or GTP (˜30 nM) as indicated. All reactions included 100 mM NaCl, 10 mM MgCl₂, 40 mM Tris-HCl (pH 7.5), and reactions were incubated at 37° C. for 1.5 hr. Reactions were terminated with the addition of 5 U of alkaline phosphatase (New England Biolabs) and incubation at 37° C. for 30 min. One microliter of each reaction was spotted onto a PEI-Cellulose F thin-layer chromatography plate (EMD Millipore), and reaction products were separated with the use of 1.5 M KH₂PO₄ (pH 3.8) as solvent. Plates were dried and radiolabeled products were detected with a phosphor screen and the Typhoon phosphorimager (GE Life Sciences).

### General Procedures for In Vitro Fluorescence Assays

In vitro fluorescence assays were carried out in a buffer containing 40 mM HEPES, pH 7.5, 125 mM KCl. Other conditions, including temperature, concentrations of MgCl₂, DFHBI (or DFHBI-1T), ligand (or cell extract) and RNA were varied in different experiments and are indicated in the figures. The RNA was renatured in buffer at 70° C. for 3 min and cooled to ambient temperature for 5 min prior to addition to the reaction solution. DFHBI (or DFHBI-1T) was added to a solution containing buffer and ligand, and then RNA was added at the end before fluorescence measurement. Binding reactions were performed either in 100 μL volumes (96-well plate) or 30 μL (384-well plate) and were incubated at the indicated temperature in either a Corning Costar 3915 96-well black plate or Greiner 781077 384-well black plate until equilibrium was reached, which typically takes 30 to 60 minutes. The fluorescence emission was measured using a Molecular Devices SpectraMax Paradigm Multi-Mode detection platform plate reader (Sunnyvale, Calif.) with the following instrument parameters (for DFHBI): 448 nm excitation, 506 nm emission. In case of DFHBI-1T 470 nm excitation and 510 nm emission was used.

Binding Affinity Analysis of 2′, 3′-cGAMP Biosensors

To measure the binding affinities of 2′, 3′-cGAMP biosensors, fluorescence assays were performed with the following conditions: 37° C., 3 mM MgCl₂, 200 nM RNA, and 10 μM DFHBI. The 2′, 3′-cGAMP concentration (c_{cGAMP}) was varied, and the fluorescence of the sample with DFHBI but no RNA was subtracted as background to determine relative fluorescence units. The ratio between the fraction of biosensor that is bound by 2′, 3′-cGAMP and the total 2′, 3′-cGAMP biosensor concentration is given as:

$$ratio bound = \frac{[ RNA \cdot cGAMP \cdot DFHBI ]}{{c}_{total RNA}} = \frac{F - {F}_{m i n}}{{F}_{ma x} - {F}_{m i n}}$$

Thus, for each concentration of 2′, 3′-cGAMP, the fluorescence of its corresponding sample should be:

$$F = \frac{[ RNA \cdot cGAMP \cdot DFHBI ]}{{c}_{total RNA}} \times ( {F}_{ma x} - {F}_{m i n} ) + {F}_{m i n}$$

where Fₘᵢₙ is the fluorescence of the sample containing no 2′,3′-cGAMP, while Fₘₐₓ is the fluorescence of the sample containing highest concentration of 2′,3′-cGAMP (100 μM). The dissociation constants K_{d} were determined by least squares fitting to the following equation in Graphpad Prism software:

$$F = \frac{[ RNA \cdot cGAMP \cdot DFHBI ]}{{c}_{total RNA}} \times ( {F}_{ma x} - {F}_{m i n} ) + {F}_{m i n} = [ \frac{\begin{matrix}{c}_{total RNA} + {K}_{d} + {c}_{cGAMP} - \\ \sqrt{{( {c}_{total RNA} + {K}_{d} + {c}_{cGAMP} )}^{2} - 4 \times {c}_{total RNA} \times {c}_{cGAMP}}\end{matrix}}{2 \times {c}_{total RNA}} ] \times ( {F}_{ma x} - {F}_{m i n} ) + {F}_{m i n}$$

cGAS Activity and Inhibition Assay

To initiate the enzyme reaction, 0.5 μL of HT-DNA was added to 2.5 μL of 6× enzyme reaction solution containing cGAS, GTP, ATP and cGAS reaction buffer. Generally, final concentrations are 100m/mL HT-DNA, 1.5 μM cGAS, 200 μM GTP, 200 μM ATP, and cGAS reaction buffer (40 mM Tris-HCl (pH 7.5), 100 mM NaCl, 10 mM MgCl₂). The reaction was incubated at 37° C. for 2 hours to allow the enzyme reaction to proceed. For the inhibition assay shown in FIG. 8, the reaction conditions are the same as described above except varied concentration of Quinacrine dihydrochloride was added. To initiate the fluorescent biosensor reaction, 3 μL of the enzyme reaction was added to 27 μL of biosensor reaction solution containing renatured RNA, DFHBI (or DFHBI-1T), and biosensor activation buffer. Final concentrations are 200 nM RNA, 10 μM DFHBI (or DFHBI-1T), 40 mM HEPES, pH 7.5, 125 mM KCl, 10 mM MgCl₂, and 0.1×cGAS reaction buffer (40 mM Tris-HCl (pH 7.5), 100 mM NaCl, 10 mM MgCl₂). In vitro fluorescence assays were conducted as described above. For the inhibition assay, due to the fluorescence of Quinacrine dihydrochloride, DFHBI-1T was used instead. Samples containing only Quinacrine dihydrochloride of varied concentration in the buffer were used for background fluorescence subtraction.

High-Throughput Screening (HTS) Assay of cGAS Activity

For HTS experiments, samples were dispensed into a Grenier 781077 384-well flat bottom black plate using an Eppendorf Repeater Xstream pipetter to dispense reaction components. Z′ scores were calculated from 16 well replicates in the 384-well plate. For the cGAS activity assay, 0.5 μL of HT-DNA or water was added to 2.5 μL of reaction solution containing cGAS, GTP, ATP and cGAS reaction buffer in each well. Final concentrations are: 100m/mL HT-DNA, 1.5 μM cGAS, 200 μM GTP, 200 μM ATP, and cGAS reaction buffer (40 mM Tris-HCl (pH 7.5), 100 mM NaCl, 10 mM MgCl₂). After 2 hrs incubation at 37° C., to initiate the fluorescent biosensor reaction, 3 μL of the reaction solution was added in each well of the 384-well plate to 27 μL of biosensor assay solution containing renatured RNA, DFHBI, and biosensor activation buffer. Final concentrations are 200 nM RNA, 10 μM DFHBI, 40 mM HEPES, pH 7.5, 125 mM KCl, 10 mM MgCl₂, and 0.1×cGAS reaction buffer (40 mM Tris-HCl (pH 7.5), 100 mM NaCl, 10 mM MgCl₂). In vitro fluorescence assays were conducted as described above.

Preparation of Cell Extract from E. coli and Human Cell Lines

Cyclic dinucleotides were extracted according to the following procedure. Cells were pelleted and frozen at −80 degree freezer overnight. Frozen cell pellets were thawed and resuspended in 1.4 mL extraction buffer (40% acetonitrile, 40% methanol and 20% ddH₂O). The resuspended cells were incubated at room temperature with mild agitation for 20 mins. After centrifugation for 5 min at 13,200 rpm, the supernatant was carefully removed and stored on ice. The remaining pellet was extracted twice more as described, with 700 μL extraction solvent each time. The combined supernatants were evaporated to dryness by rotary evaporation and the dried material was resuspended in 300 μL ddH₂O. The extract was filtered through a 3 kDa MW CO Amicon Ultra-4 Protein Concentrator (Millipore) to remove majority of the proteins and thereby enrich small molecule metabolite and secondary messengers. For bacterial cells, the filtered extract was used immediately or stored at −20° C. Human cell extracts were further concentrated to a final volume of 30 μL using vacuum concentrator and then either used immediately or stored at −20° C.

In Vitro Fluorescence Assay with Cell Extracts

To initiate the fluorescent biosensor reaction, 3 μL of the bacterial cell extract was added to 27 μL of biosensor reaction solution containing renatured RNA, DFHBI (or DFHBI-1T), and biosensor activation buffer. For the dose dependent mammalian cell extract experiments, 2 uL (1× cell lysate), 5 uL (2.5× cell lysate) and 10 uL (5× cell lysate) of cell extracts (stimulated or non-stimulated) were added to a final volume of 30 μL reaction solution. Final concentrations are 200 nM RNA, 10 μM DFHBI (or DFHBI-1T), 40 mM HEPES, pH 7.5, 125 mM KCl, 10 mM MgCl₂. In vitro fluorescence assays were conducted as described above. For the experiments with SVPD, active or heat inactivated SVPD (200 μU) was added to 2 uL (1× cell lysate) of cell extract and kept at 37° C. for 1 hour. This reaction was stopped by heating at 95° C. for 5 mins, followed by rapid cooling on ice. Then it was added to a final volume of 30 μL reaction solution as mentioned previously. For the doping experiment, a range of known conc. of commercial 2′,3′-cGAMP were added to 1 uL non stimulated cell extract followed by addition to a final volume of 30 μL reaction solution as mentioned previously.

Calculating the Concentration of 2′, 3′-cGAMP in Mammalian Cell Extract

To obtain a standard curve for quantifying 2′,3′-cGAMP using the biosensor, 6×10⁶ non-stimulated cells were lysed, extracted and concentrated to give 20 μL cell extract. Then, a range of known concentration of commercial 2′,3′-cGAMP were doped with 2 μL non-stimulated cell extract into a solution with final volume of 25 μL for fluorescence measurement. From the linear regression of the standard curve, shown in FIG. 4F, fitting the standard curve to equation Y=mX+C gave 1/m=1.67*10⁻⁵ μM and C=150517±22135.

For measuring the average amount of 2′,3′-cGAMP in each dsDNA-stimulated cell, 6×10⁶ dsDNA stimulated cells were lysed, extracted and concentrated to give 20 μL cell extract, and 2 μL of the cell extract was added to a solution with final volume of 25 μL for fluorescence measurement. The fluorescence of this solution was measured to be 239874 (FIG. 5E, in duplicate).

Thus, the concentration of 2′,3′-cGAMP in the 25 μL measurement solution was calculated to be:

$$c ( cGAMP ) = \frac{Y - C}{m} = ( 239874 - 150517 ) \times ( 1.67 \times {10}^{- 5} ) μM = 1.5 μM$$

Then, the amount of 2′,3′-cGAMP in the 2 μL cell extract was:

n(cGAMP)=c(cGAMP)×volume=1.5 μM×25 μL=37 pmol

Lastly, the amount of 2′,3′-cGAMP per cell was determined to be:

$$n ( cGAMP per cell ) = \frac{n ( cGAMP )}{number of cells} = \frac{37 pmol \times 10}{6 \times {10}^{6}} \approx 6 \times {10}^{- 17} mol = 60 attomol$$

Calculating the Concentration of 2′, 3′-cGAMP in Stimulated E. coli Cells

Escherichia coli cells expressing empty pCOLA vector and cGAS expressing pCOLA vector were induced with IPTG and four hours post induction the cells were pelleted and dry weight was measured. Cell extracts were made from the cell pallets as described previously. To obtain a standard curve for quantifying 2′,3′-cGAMP, a range of known concentration of commercial 2′,3′-cGAMP were doped with empty pCOLA expressing cell extract to a final volume of 20 μL for LC-MS based quantification. This was run in LC-MS and 675 m/z trace (corresponding to 2′,3′-cGAMP) was extracted. From the linear regression of the standard curve, shown in FIG. S5, fitting the standard curve to equation Y=mX+C gave

Y=2.49X−7.46

The dry weight of the cGAS expressing cell pallet was 0.75g. This was lysed, extracted and concentrated to give 250 μL cell extract, and 20 μL of the cell extract was loaded in LC-MS.

Thus, the concentration of 2′,3′-cGAMP in the 20 μL measurement solution was calculated to be:

$$c ( cGAMP ) = \frac{Y - C}{m} = \frac{3372 + 7.46}{2.49 \times 1000} mM = 1.36 mM$$

The amount of 2′,3′-cGAMP in the 250 μL cell extract was:

n(cGAMP)=c(cGAMP)×volume=1.36 mM×250 μL=0.34 μmol

The dry weight of single E. coli cell is 10⁻¹² g, which gives:

$$number of cells = \frac{cell pallet weight}{dry weight of single cell} = \frac{0.75}{{10}^{- 12}} \approx 0.75 \times {10}^{12}$$

The amount of 2,3-cGAMP per cell was determined to be:

$$n ( cGAMP per cell ) = \frac{n ( cGAMP )}{number of cells} = \frac{0.34 μmol}{0.75 \times {10}^{12}} \approx 0.45 \times {10}^{- 18} mol = 0.45 attomol$$

The volume of E. coli cell is 10⁻¹⁵ L. The concentration of 2′,3′-cGAMP in cGAS expressing E. coli cell was determined by:

$$\begin{matrix}[ cGAMP per cell ] = \frac{n ( cGAMP per cell )}{cell volume} \\ = \frac{0.45 \times {10}^{- 18} mol}{{10}^{- 15} L} \approx 0.45 \times {10}^{- 3} M \\ = 0.45 mM\end{matrix}$$

### In Vivo Fluorescence Assays by Flow Cytometry

Preparation of cell samples for flow cytometry was carried out by inoculating 3 mL of LB media (containing carbenicillin and kanamycin) with 150 μL of an overnight grown culture of BL21 star cells harboring both a pET31b plasmid encoding RNA-based biosensor and a pCOLA plasmid encoding cyclic-di-nucleotide synthetase. Cells were grown aerobically to an OD₆₀₀˜0.3-0.5, then induced with 1 mM IPTG at 37° C. for 4 hrs. Cell density was measured by OD₆₀₀, and diluted in PBS media containing 100 μM DFHBI-1T to a concentration that gives a rate of around 1000-2000 events per second on flow cytometer. Cellular fluorescence was measured for 30,000 cells using a Life Technologies Attune N×T Acoustic Focusing Cytometer.

TABLE 1
GEMM-II riboswitch-based biosensors sequences

| Name | Sequences |
|---|---|
| Ca-2 P1-6 | GATGTAACTGAATGAAATGGTGAAGGACGGGTCCA [symbol] AAACAATGATGAATTTCT |
| | TTAAATTGGGCACTTGAGAAATTTTGAGTTAGTAGTGCAACCGA [symbol] TTGTTGAGTA |
| | GAGTGTGAGCTCCGTAACTAGTTACATC (SEQ ID NO: 1) |
| Ca-2 P1-5 | GATGTAACTGAATGAAATGGTGAAGGACGGGTCCA [symbol] AAACAATGATGAATTTCTT |
| | TAAATTGGGCACTTGAGAAATTTTGAGTTAGTAGTGCAACCGA [symbol] TTGTTGAGTAGA |
| | GTGTGAGCTCCGTAACTAGTTACATC (SEQ ID NO: 2) |
| Ca-2 P1-4 | GATGTAACTGAATGAAATGGTGAAGGACGGGTCCA [symbol] AAACAATGATGAATTTCTTTA |
| | AATTGGGCACTTGAGAAATTTTGAGTTAGTAGTGCAACCGA [symbol] TTGTTGAGTAGAGTG |
| | TGAGCTCCGTAACTAGTTACATC (SEQ ID NO: 3) |
| Ca-2 P1-3 | GATGTAACTGAATGAAATGGTGAAGGACGGGTCCA [symbol] AAACAATGATGAATTTCTTTAA |
| | ATTGGGCACTTGAGAAATTTTGAGTTAGTAGTGCAACCGA [symbol] TTGTTGAGTAGAGTGTG |
| | AGCTCCGTAACTAGTTACATC (SEQ ID NO: 4) |
| Cd1-1 P1-5 | GATGTAACTGAATGAAATGGTGAAGGACGGGTCCA [symbol] AAACTGTGAAGTATATCTTA |
| | AACCTGGGCACTTAAAAGATATATGGAGTTAGTAGTGCAACCTG [symbol] TTGTTGAGTAG |
| | AGTGTGAGCTCCGTAACTAGTTACATC (SEQ ID NO: 5) |
| Cd1-1 P1-4 | GATGTAACTGAATGAAATGGTGAAGGACGGGTCCA [symbol] AAACTGTGAAGTATATCTTAA |
| | ACCTGGGCACTTAAAAGATATATGGAGTTAGTAGTGCAACCTG [symbol] TTGTTGAGTAGAG |
| | TGTGAGCTCCGTAACTAGTTACATC (SEQ ID NO: 6) |
| Cd1-1 P1-3 | GATGTAACTGAATGAAATGGTGAAGGACGGGTCCA [symbol] AAACTGTGAAGTATATCTTAAA |
| | CCTGGGCACTTAAAAGATATATGGAGTTAGTAGTGCAACCTG [symbol] TTGTTGAGTAGAGTG |
| | TGAGCTCCGTAACTAGTTACATC (SEQ ID NO: 7) |
| Cd2-1 P1-6 | GATGTAACTGAATGAAATGGTGAAGGACGGGTCCA [symbol] AAACTGAGAAGTATATCTT |
| | ATTATTGGGCATCTGGAGATATATGGAGTTAGTGGTGCAACCGG [symbol] TTGTTGAGT |
| | AGAGTGTGAGCTCCGTAACTAGTTACATC (SEQ ID NO: 8) |
| Cd2-1 P1-5 | GATGTAACTGAATGAAATGGTGAAGGACGGGTCCA [symbol] AAACTGAGAAGTATATCTTA |
| | TTATTGGGCATCTGGAGATATATGGAGTTAGTGGTGCAACCGG [symbol] TTGTTGAGTAG |
| | AGTGTGAGCTCCGTAACTAGTTACATC (SEQ ID NO: 9) |
| Cd2-1 P1-4 | GATGTAACTGAATGAAATGGTGAAGGACGGGTCCA [symbol] AAACTGAGAAGTATATCTTAT |
| | TATTGGGCATCTGGAGATATATGGAGTTAGTGGTGCAACCGG [symbol] TTGTTGAGTAGAG |
| | TGTGAGCTCCGTAACTAGTTACATC (SEQ ID NO: 10) |
| Cd2-1 P1-3 | GATGTAACTGAATGAAATGGTGAAGGACGGGTCCA [symbol] AAACTGAGAAGTATATCTTATT |
| | ATTGGGCATCTGGAGATATATGGAGTTAGTGGTGCAACCGG [symbol] TTGTTGAGTAGAGTGT |
| | GAGCTCCGTAACTAGTTACATC (SEQ ID NO: 11) |
| Bh P1-6 (WT) | GATGTAACTGAATGAAATGGTGAAGGACGGGTCCA [symbol] AAGCAACGAAGCATAGCC |
| | TTTATATGGACACTTGGGTTATGTGGAGCTACTAGTGTAACCGG [symbol] TTGTTGAG |
| | TAGAGTGTGAGCTCCGTAACTAGTTACATC (SEQ ID NO: 12) |
| Bh P1-5 | GATGTAACTGAATGAAATGGTGAAGGACGGGTCCA [symbol] AAGCAACGAAGCATAGCCT |
| | TTATATGGACACTTGGGTTATGTGGAGCTACTAGTGTAACCGG [symbol] TTGTTGAGTA |
| | GAGTGTGAGCTCCGTAACTAGTTACATC (SEQ ID NO: 13) |
| Bh P1-4 | GATGTAACTGAATGAAATGGTGAAGGACGGGTCCA [symbol] AAGCAACGAAGCATAGCCTTT |
| | ATATGGACACTTGGGTTATGTGGAGCTACTAGTGTAACCGG [symbol] TTGTTGAGTAGAGTG |
| | TGAGCTCCGTAACTAGTTACATC (SEQ ID NO: 14) |
| Bh P1-3 | GATGTAACTGAATGAAATGGTGAAGGACGGGTCCA [symbol] AAGCAACGAAGCATAGCCTTTA |
| | TATGGACACTTGGGTTATGTGGAGCTACTAGTGTAACCGG [symbol] TTGTTGAGTAGAGTGTG |
| | AGCTCCGTAACTAGTTACATC (SEQ ID NO: 15) |
| Bh P1-5 delC | GATGTAACTGAATGAAATGGTGAAGGACGGGTCCA [symbol] AAGCAACGAAGCATAGCCT |
| | TTATATGGACACTTGGGTTATGTGGAGCTACTAGTGTAACCGG [symbol] TCTTTGTTGAGTAG |
| | AGTGTGAGCTCCGTAACTAGTTACATC (SEQ ID NO: 16) |
| Bh P1-6 | GATGTAACTGAATGAAATGGTGAAGGACGGGTCCA [symbol] AAGCAACGAAGCATAGCC |
| G103A | TTTATATGGACACTTGGGTTATGTGGAGCTACTAGTGTAACC [symbol] G [symbol] TTGTTGAG |
| | TAGAGTGTGAGCTCCGTAACTAGTTACATC (SEQ ID NO: 18) |
| Bh P1-6 A99G | GATGTAACTGAATGAAATGGTGAAGGACGGGTCCA [symbol] AAGCAACGAAGCATAGCC |
| | TTTATATGGACACTTGGGTTATGTGGAGCTACTAGTGT [symbol] ACCGG [symbol] TTGTTGAG |
| | TAGAGTGTGAGCTCCGTAACTAGTTACATC (SEQ ID NO: 19) |
| Bh P1-6 A99C | GATGTAACTGAATGAAATGGTGAAGGACGGGTCCA [symbol] AAGCAACGAAGCATAGCC |
| | TTTATATGGACACTTGGGTTATGTGGAGCTACTAGTGT [symbol] ACCGG [symbol] TTGTTGAG |
| | TAGAGTGTGAGCTCCGTAACTAGTTACATC (SEQ ID NO: 20) |
| Bh P1-6 | GATGTAACTGAATGAAATGGTGAAGGACGGGTCCA [symbol] AAGCAACGAAGCATAGCC |
| G103C | TTTATATGGACACTTGGGTTATGTGGAGCTACTAGTGTAACC [symbol] G [symbol] TTGTTGAG |
| | TAGAGTGTGAGCTCCGTAACTAGTTACATC (SEQ ID NO: 21) |
| Bh P1-6 | GATGTAACTGAATGAAATGGTGAAGGACGGGTCCA [symbol] AAGCAACGAAGCATAGCC |
| G103U | TTTATATGGACACTTGGGTTATGTGGAGCTACTAGTGTAACC [symbol] G [symbol] TTGTTGAG |
| | TAGAGTGTGAGCTCCGTAACTAGTTACATC (SEQ ID NO: 22) |
| Bh P1-6 A99G | GATGTAACTGAATGAAATGGTGAAGGACGGGTCCA [symbol] AAGCAACGAAGCATAGCC |
| U67C | TTTATA [symbol] GGACACTTGGGTTATGTGGAGCTACTAGTGT [symbol] ACCGG [symbol] TTGTTGAG |
| | TAGAGTGTGAGCTCCGTAACTAGTTACATC (SEQ ID NO: 23) |
| Spinach2 | CCAAGTAATACGACTCACTATAG GATGTAACTGAATGAAATGGTGAAG (SEQ ID |
| forward primer | NO: 24) |
| (with T7 | |
| promoter) | |
| Spinach2 | GATGTAACTAGTTACGGAGCTCAC (SEQ ID NO: 25) |
| reverse primer | |

In the biosensors sequence, Spinach2 sequence is underlined, sequence of transducer stem between Spinach2 aptamer and GEMM-II riboswtich is bolded and itatic, and mutated nucleotides are bolded, underlined and itatic.

Ca: Bacteria Firmicutes Clostridia Clostridiales Clostridiaceae Clostridium acetobutylicum ATCC 824
Cd1: Bacteria Firmicutes Clostridia Clostridiales Clostridiaceae Clostridium difficile 630
Cd2: Bacteria Firmicutes Clostridia Clostridiales Clostridiaceae Clostridium difficile QCD-32g58
Bh: Bacteria Firmicutes Bacillales Bacillaceae Bacillus halodurans C-125

TABLE 2
Comparison of GEMM-II biosensors binding affinities and fluorescent turn-on properties

| Kd/μM | | | | | | |
|---|---|---|---|---|---|---|
| (fold turn-on^{a}) | Bh P1-5delC | | Bh P1-6 WT | | Bh P1-6 G103A | |
| c-di-GMP | 0.021 ± 0.008 | (—) | 0.056 ± 0.012 | (13.1x) | >7.4 | (8.2x) |
| 3′,3′-cGAMP | — | (—) | >11 | (10.8x) | 0.8 ± 0.1 | (9.6x) |
| 2′,3′-cGAMP | 13.4 ± 0.9 | (5.1x) | — | (—) | >65 | (10.3) |

^{a}Under condition of 200 nM (2', 3'-cGAMP)/100 nM (c-di-GMP, 3',3'-cGAMP) RNA, 100 μM ligand in a 387-well plate.

Notwithstanding the appended claims, the disclosure set forth herein is also described by the following clauses:

Clause 1. A single stranded nucleic acid, comprising: a GEMM-II riboswitch domain that specifically binds 2′, 3′-cGAMP; and a signaling chromophore-binding Spinach2 aptamer domain that is operably connected to the GEMM-II riboswitch domain via a transducer stem.

Clause 2. The nucleic acid of clause 1, wherein the transducer stem comprises the sequences:

5′-AAUAGGG-3′;
and
3′-UUCCUCCC-5′.

Clause 3. The nucleic acid of any one of clauses 1-2, wherein the GEMM-II riboswitch domain has 80% or greater nucleotide sequence identity to the riboswitch domain of SEQ ID NO:17 in FIG. 2.

Clause 4. The nucleic acid of any one of clauses 1-3, wherein the Spinach2 aptamer domain has 80% or greater nucleotide sequence identity to the Spinach2 aptamer domain of SEQ ID NO:17 in FIG. 2.

Clause 5. A nucleic acid construct encoding the single stranded nucleic acid of any one of clauses 1-4.

Clause 6. A host cell comprising the nucleic acid construct of clause 5.

Clause 7. A biosensor, comprising:

a single stranded nucleic acid comprising:
a GEMM-II riboswitch domain that specifically binds 2′, 3′-cGAMP; and
a signaling chromophore-binding Spinach2 aptamer domain that is operably connected to the GEMM-II riboswitch domain via a transducer stem; and

a signaling chromophore specifically bound to the Spinach2 aptamer domain;

wherein the sensor is configured to fluorescently activate the signaling chromophore upon specific binding of 2′, 3′-cGAMP to the GEMM-II riboswitch domain.

Clause 8. The biosensor of clause 7, wherein the fluorescence activation of the signaling chromophore is by 50% or more.

Clause 9. The biosensor of any one of clauses 7-8, wherein the biosensor is configured to specifically bind 2′, 3′-cGAMP with at least 10-fold stronger affinity over ATP or GTP.

Clause 10. The biosensor of any one of clauses 7-9, wherein the transducer stem comprises the sequences: 5′-AAUAGGG-3′; and 3′-UUCCUCCC-5′.

Clause 11. The biosensor of any one of clauses 7-10, wherein the GEMM-II riboswitch domain has 80% or greater nucleotide sequence identity to the riboswitch domain of SEQ ID NO:17 in FIG. 2.

Clause 12. The biosensor of any one of clauses 7-11, wherein the Spinach2 aptamer domain has 80% or greater nucleotide sequence identity to the Spinach2 aptamer domain of SEQ ID NO:17 in FIG. 2.

Clause 13. The biosensor of any one of clauses 7-12, wherein the Spinach2 aptamer domain is comprises an open P1 stem having 5′ and 3′ terminals.

Clause 14. The biosensor of any one of clauses 7-13, comprising the single stranded nucleic acid of clause 1.

Clause 15. A method for determining the level of 2′, 3′-cGAMP in a sample, the method comprising:

contacting the sample with a biosensor according to clause 7; and

detecting fluorescence from the biosensor thereby determining the level of 2′,3′-cGAMP in the sample.

Clause 16. The method of clause 15, wherein the determined level of 2′,3′-cGAMP in the sample is independent of the level of ATP or GTP in the sample.

Clause 17. The method of any one of clauses 15-16, further comprising determining a cGAS activity of the sample based on the determined level of 2′, 3′-cGAMP.

Clause 18. The method of any one of clauses 15-17, wherein the sample is a cellular sample.

Clause 19. The method of any one of clauses 15-18, further comprising contacting the sample with a candidate cGAS-modulating agent and determining whether the agent inhibits or activates cGAS in the sample.

Clause 20. A method for determining level of cGAS activity in a cell, the method comprising:

contacting the cell with a single stranded nucleic acid according to any one of clauses 1-5 and a signaling chromophore to produce a 2′, 3′-cGAMP biosensor in situ; and

detecting fluorescence from the signaling chromophore of the 2′, 3′-cGAMP biosensor thereby determining the level of cGAS activity in the cell.

Clause 21. The method of clause 20, wherein the single stranded nucleic acid is expressed by the cell.

Clause 22. The method of any one of clauses 20-21, further comprising monitoring fluorescence of the signaling chromophore upon application of a stimulus to the cell.

Clause 23. A kit comprising:

a single stranded nucleic acid of any one of clauses 1-5 or a nucleic acid construct encoding the single stranded nucleic acid of any one of clauses 1-5; and

one or more components selected from a signaling chromophore, a promoter, a cell, a cloning vector and an expression cassette.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A single stranded nucleic acid, comprising:
a GEMM-II riboswitch domain that specifically binds 2′, 3′-cGAMP; and
a signaling chromophore-binding Spinach2 aptamer domain that is operably connected to the GEMM-II riboswitch domain via a transducer stem.

2. The nucleic acid of claim 1, wherein the transducer stem comprises the sequences:
5′-AAUAGGG-3′;
and
3′-UUCCUCCC-5′.

3. The nucleic acid of claim 1, wherein the GEMM-II riboswitch domain has 80% or greater nucleotide sequence identity to the riboswitch domain of SEQ ID NO:17 in FIG. 2.

4. The nucleic acid of claim 1, wherein the Spinach2 aptamer domain has 80% or greater nucleotide sequence identity to the Spinach2 aptamer domain of SEQ ID NO:17 in FIG. 2.

5. A nucleic acid construct comprising a nucleotide sequence encoding the single stranded nucleic acid of claim 1.

6. A host cell comprising the nucleic acid construct of claim 5.

7. A biosensor, comprising:
a) a single stranded nucleic acid comprising:
a GEMM-II riboswitch domain that specifically binds 2′, 3′-cGAMP; and
a signaling chromophore-binding Spinach2 aptamer domain that is operably connected to the GEMM-II riboswitch domain via a transducer stem; and
b) a signaling chromophore specifically bound to the Spinach2 aptamer domain;
wherein the sensor is configured to fluorescently activate the signaling chromophore upon specific binding of 2′, 3′-cGAMP to the GEMM-II riboswitch domain.

8. The biosensor of claim 7, wherein the fluorescence activation of the signaling chromophore is by 50% or more; and the biosensor is configured to specifically bind 2′, 3′-cGAMP with at least 10-fold stronger affinity over ATP or GTP.

9. The biosensor of claim 7, wherein the transducer stem comprises the sequences:
5′-AAUAGGG-3′;
and
3′-UUCCUCCC-5′.

10. The biosensor of claim 7, wherein the GEMM-II riboswitch domain has 80% or greater nucleotide sequence identity to the riboswitch domain of SEQ ID NO:17 in FIG. 2.

11. The biosensor of claim 7, wherein the Spinach2 aptamer domain has 80% or greater nucleotide sequence identity to the Spinach2 aptamer domain of SEQ ID NO:17 in FIG. 2.

12. The biosensor of claim 7, wherein the Spinach2 aptamer domain is comprises an open P1 stem having 5′ and 3′ terminals.

13. A method for determining the level of 2′, 3′-cGAMP in a sample, the method comprising:
contacting the sample with a biosensor according to claim 7; and
detecting fluorescence from the biosensor thereby determining the level of 2′,3′-cGAMP in the sample.

14. The method of claim 13, wherein the determined level of 2′,3′-cGAMP in the sample is independent of the level of ATP or GTP in the sample.

15. The method of claim 13, further comprising determining a cGAS activity of the sample based on the determined level of 2′, 3′-cGAMP.

16. The method of claim 13, wherein the sample is a cellular sample.

17. The method of claim 16, further comprising contacting the sample with a candidate cGAS-modulating agent and determining whether the agent inhibits or activates cGAS in the sample.

18. A method for determining level of cGAS activity in a cell, the method comprising:
contacting the cell with a single stranded nucleic acid according to claim 1 and a signaling chromophore to produce a 2′, 3′-cGAMP biosensor in situ; and
detecting fluorescence from the signaling chromophore of the 2′, 3′-cGAMP biosensor thereby determining the level of cGAS activity in the cell.

19. The method of claim 18, wherein the single stranded nucleic acid is expressed by the cell.


SEQUENCE LISTING (34 sequences)

SEQ ID NO: 1 - DNA, 148 bp, Artificial sequence
  synthetic nucleic acid
gatgtaactgaatgaaatggtgaaggacgggtccatgtttggaaacaatgatgaatttctttaaattgggcacttgagaaattttgagttagtagtgcaaccgaccaacgttgttgagtagagtgtgagctccgtaactagttacatc

SEQ ID NO: 2 - DNA, 146 bp, Artificial sequence
  synthetic nucleic acid
gatgtaactgaatgaaatggtgaaggacgggtccagtttggaaacaatgatgaatttctttaaattgggcacttgagaaattttgagttagtagtgcaaccgaccaacttgttgagtagagtgtgagctccgtaactagttacatc

SEQ ID NO: 3 - DNA, 143 bp, Artificial sequence
  synthetic nucleic acid
gatgtaactgaatgaaatggtgaaggacgggtccattggaaacaatgatgaatttctttaaattgggcacttgagaaattttgagttagtagtgcaaccgaccaattgttgagtagagtgtgagctccgtaactagttacatc

SEQ ID NO: 4 - DNA, 141 bp, Artificial sequence
  synthetic nucleic acid
gatgtaactgaatgaaatggtgaaggacgggtccatggaaacaatgatgaatttctttaaattgggcacttgagaaattttgagttagtagtgcaaccgaccattgttgagtagagtgtgagctccgtaactagttacatc

SEQ ID NO: 5 - DNA, 147 bp, Artificial sequence
  synthetic nucleic acid
gatgtaactgaatgaaatggtgaaggacgggtccatatagaaactgtgaagtatatcttaaacctgggcacttaaaagatatatggagttagtagtgcaacctgctatattgttgagtagagtgtgagctccgtaactagttacatc

SEQ ID NO: 6 - DNA, 145 bp, Artificial sequence
  synthetic nucleic acid
gatgtaactgaatgaaatggtgaaggacgggtccaatagaaactgtgaagtatatcttaaacctgggcacttaaaagatatatggagttagtagtgcaacctgctatttgttgagtagagtgtgagctccgtaactagttacatc

SEQ ID NO: 7 - DNA, 143 bp, Artificial sequence
  synthetic nucleic acid
gatgtaactgaatgaaatggtgaaggacgggtccatagaaactgtgaagtatatcttaaacctgggcacttaaaagatatatggagttagtagtgcaacctgctattgttgagtagagtgtgagctccgtaactagttacatc

SEQ ID NO: 8 - DNA, 149 bp, Artificial sequence
  synthetic nucleic acid
gatgtaactgaatgaaatggtgaaggacgggtccattttagaaactgagaagtatatcttattattgggcatctggagatatatggagttagtggtgcaaccggctatgaattgttgagtagagtgtgagctccgtaactagttacatc

SEQ ID NO: 9 - DNA, 147 bp, Artificial sequence
  synthetic nucleic acid
gatgtaactgaatgaaatggtgaaggacgggtccatttagaaactgagaagtatatcttattattgggcatctggagatatatggagttagtggtgcaaccggctatgattgttgagtagagtgtgagctccgtaactagttacatc

SEQ ID NO: 10 - DNA, 145 bp, Artificial sequence
  synthetic nucleic acid
gatgtaactgaatgaaatggtgaaggacgggtccattagaaactgagaagtatatcttattattgggcatctggagatatatggagttagtggtgcaaccggctatgttgttgagtagagtgtgagctccgtaactagttacatc

SEQ ID NO: 11 - DNA, 142 bp, Artificial sequence
  synthetic nucleic acid
gatgtaactgaatgaaatggtgaaggacgggtccatagaaactgagaagtatatcttattattgggcatctggagatatatggagttagtggtgcaaccggctattgttgagtagagtgtgagctccgtaactagttacatc

SEQ ID NO: 12 - DNA, 150 bp, Artificial sequence
  synthetic nucleic acid
gatgtaactgaatgaaatggtgaaggacgggtccaaatagggaagcaacgaagcatagcctttatatggacacttgggttatgtggagctactagtgtaaccggccctccttttgttgagtagagtgtgagctccgtaactagttacatc

SEQ ID NO: 13 - DNA, 148 bp, Artificial sequence
  synthetic nucleic acid
gatgtaactgaatgaaatggtgaaggacgggtccaatagggaagcaacgaagcatagcctttatatggacacttgggttatgtggagctactagtgtaaccggccctcctttgttgagtagagtgtgagctccgtaactagttacatc

SEQ ID NO: 14 - DNA, 143 bp, Artificial sequence
  synthetic nucleic acid
gatgtaactgaatgaaatggtgaaggacgggtccaagggaagcaacgaagcatagcctttatatggacacttgggttatgtggagctactagtgtaaccggccctttgttgagtagagtgtgagctccgtaactagttacatc

SEQ ID NO: 15 - DNA, 141 bp, Artificial sequence
  synthetic nucleic acid
gatgtaactgaatgaaatggtgaaggacgggtccagggaagcaacgaagcatagcctttatatggacacttgggttatgtggagctactagtgtaaccggcccttgttgagtagagtgtgagctccgtaactagttacatc

SEQ ID NO: 16 - DNA, 147 bp, Artificial sequence
  synthetic nucleic acid
gatgtaactgaatgaaatggtgaaggacgggtccaatagggaagcaacgaagcatagcctttatatggacacttgggttatgtggagctactagtgtaaccggccctctttgttgagtagagtgtgagctccgtaactagttacatc

SEQ ID NO: 17 - DNA, 2 bp, Artificial sequence
  synthetic nucleic acid
ga

SEQ ID NO: 18 - DNA, 150 bp, Artificial sequence
  synthetic nucleic acid
gatgtaactgaatgaaatggtgaaggacgggtccaaatagggaagcaacgaagcatagcctttatatggacacttgggttatgtggagctactagtgtaaccagccctccttttgttgagtagagtgtgagctccgtaactagttacatc

SEQ ID NO: 19 - DNA, 150 bp, Artificial sequence
  synthetic nucleic acid
gatgtaactgaatgaaatggtgaaggacgggtccaaatagggaagcaacgaagcatagcctttatatggacacttgggttatgtggagctactagtgtgaccggccctccttttgttgagtagagtgtgagctccgtaactagttacatc

SEQ ID NO: 20 - DNA, 150 bp, Artificial sequence
  synthetic nucleic acid
gatgtaactgaatgaaatggtgaaggacgggtccaaatagggaagcaacgaagcatagcctttatatggacacttgggttatgtggagctactagtgtcaccggccctccttttgttgagtagagtgtgagctccgtaactagttacatc

SEQ ID NO: 21 - DNA, 150 bp, Artificial sequence
  synthetic nucleic acid
gatgtaactgaatgaaatggtgaaggacgggtccaaatagggaagcaacgaagcatagcctttatatggacacttgggttatgtggagctactagtgtaacccgccctccttttgttgagtagagtgtgagctccgtaactagttacatc

SEQ ID NO: 22 - DNA, 150 bp, Artificial sequence
  synthetic nucleic acid
gatgtaactgaatgaaatggtgaaggacgggtccaaatagggaagcaacgaagcatagcctttatatggacacttgggttatgtggagctactagtgtaacctgccctccttttgttgagtagagtgtgagctccgtaactagttacatc

SEQ ID NO: 23 - DNA, 150 bp, Artificial sequence
  synthetic nucleic acid
gatgtaactgaatgaaatggtgaaggacgggtccaaatagggaagcaacgaagcatagcctttatacggacacttgggttatgtggagctactagtgtgaccggccctccttttgttgagtagagtgtgagctccgtaactagttacatc

SEQ ID NO: 24 - DNA, 48 bp, Artificial sequence
  synthetic nucleic acid
ccaagtaatacgactcactataggatgtaactgaatgaaatggtgaag

SEQ ID NO: 25 - DNA, 24 bp, Artificial sequence
  synthetic nucleic acid
gatgtaactagttacggagctcac

SEQ ID NO: 26 - RNA, 75 nt, Artificial sequence
  synthetic nucleic acid
  misc_feature (1)..(5): N can be any nucleotide.
  misc_feature (6)..(6): N is A or G.
  misc_feature (9)..(10): N is A or G.
  misc_feature (12)..(13): N is any nucleotide.
  misc_feature (17)..(18): N is any nucleotide.
  misc_feature (19)..(19): N is C or U.
  misc_feature (20)..(20): N is A or G.
  misc_feature (21)..(21): N is C or U.
  misc_feature (22)..(24): N is any nucleotide.
  misc_feature (25)..(25): N is C or U.
  misc_feature (27)..(27): N may be 1 to 3 nucleotides. N is any nucleotide.
  misc_feature (29)..(29): N is C or U.
  misc_feature (30)..(30): N is any nucleotide.
  misc_feature (34)..(34): N is A or G.
  misc_feature (38)..(38): N is C or U.
  misc_feature (40)..(40): N may be 1 to 22 nucleotides. N is any nucleotide.
  misc_feature (41)..(41): N is A or G.
  misc_feature (42)..(44): N is any nucleotide.
  misc_feature (45)..(45): N is A or G.
  misc_feature (46)..(46): N is C or U.
  misc_feature (47)..(47): N is A or G.
  misc_feature (48)..(48): N is any nucleotide.
  misc_feature (49)..(49): N may be 1 to 2 nucleotides. N is any nucleotide.
  misc_feature (53)..(54): N is C or U.
  misc_feature (58)..(58): N is A or G.
  misc_feature (68)..(68): N is A or G.
  misc_feature (70)..(70): N is C or U.
  misc_feature (71)..(75): N is any nucleotide.
nnnnnnganncnnugannnnnnnnnunannuggncacnunnnnnnnnnngagnnagungugcaaccgncnnnnnn

SEQ ID NO: 27 - RNA, 77 nt, Artificial sequence
  synthetic nucleic acid
aauagggaagcaacgaagcauagccuuuauauggacacuuggguuauguggagcuacuaguguaaccggcccuccuu

SEQ ID NO: 28 - RNA, 62 nt, Artificial sequence
  synthetic nucleic acid
  misc_feature (2)..(3): N is A or G.
  misc_feature (5)..(6): N is any nucleotide.
  misc_feature (10)..(11): N is any nucleotide.
  misc_feature (12)..(12): N is C or U.
  misc_feature (13)..(13): N is A or G.
  misc_feature (14)..(14): N is C or U.
  misc_feature (15)..(17): N is any nucleotide.
  misc_feature (18)..(18): N is C or U.
  misc_feature (23)..(23): N is C or U.
  misc_feature (24)..(24): N is any nucleotide.
  misc_feature (28)..(28): N is A or G.
  misc_feature (32)..(32): N is C or U.
  misc_feature (47)..(48): N is C or U.
  misc_feature (52)..(52): N is A or G.
  misc_feature (62)..(62): N is A or G.
anncnnugannnnnnnnnuuuannuggncacnuggguuauguggagnnagungugcaaccgn

SEQ ID NO: 29 - RNA, 62 nt, Artificial sequence
  synthetic nucleic acid
  misc_feature (2)..(3): N is A or G.
  misc_feature (5)..(6): N is any nucleotide.
  misc_feature (10)..(11): N is any nucleotide.
  misc_feature (12)..(12): N is C or U.
  misc_feature (13)..(13): N is A or G.
  misc_feature (14)..(14): N is C or U.
  misc_feature (15)..(17): N is any nucleotide.
  misc_feature (18)..(18): N is C or U.
  misc_feature (23)..(23): N is C or U.
  misc_feature (24)..(24): N is any nucleotide.
  misc_feature (28)..(28): N is A or G.
  misc_feature (32)..(32): N is C or U.
  misc_feature (47)..(48): N is C or U.
  misc_feature (52)..(52): N is A or G.
  misc_feature (62)..(62): N is A or G.
anncnnugannnnnnnnnuuuannuggncacnuggguuauguggagnnagungugcaaccan

SEQ ID NO: 30 - RNA, 11 nt, Artificial sequence
  synthetic nucleic acid
guagaguguga

SEQ ID NO: 31 - RNA, 35 nt, Artificial sequence
  synthetic nucleic acid
gauguaacugaaugaaauggugaaggacgggucca

SEQ ID NO: 32 - RNA, 38 nt, Artificial sequence
  synthetic nucleic acid
uuguugaguagagugugagcuccguaacuaguuacauc

SEQ ID NO: 33 - RNA, 62 nt, Artificial sequence; identical to SEQ ID NO: 28
  synthetic nucleic acid
  misc_feature (2)..(3): N is A or G.
  misc_feature (5)..(6): N is any nucleotide.
  misc_feature (10)..(11): N is any nucleotide.
  misc_feature (12)..(12): N is C or U.
  misc_feature (13)..(13): N is A or G.
  misc_feature (14)..(14): N is C or U.
  misc_feature (15)..(17): N is any nucleotide.
  misc_feature (18)..(18): N is C or U.
  misc_feature (23)..(23): N is C or U.
  misc_feature (24)..(24): N is any nucleotide.
  misc_feature (28)..(28): N is A or G.
  misc_feature (32)..(32): N is C or U.
  misc_feature (47)..(48): N is C or U.
  misc_feature (52)..(52): N is A or G.
  misc_feature (62)..(62): N is A or G.

SEQ ID NO: 34 - RNA, 62 nt, Artificial sequence; identical to SEQ ID NO: 29
  synthetic nucleic acid
  misc_feature (2)..(3): N is A or G.
  misc_feature (5)..(6): N is any nucleotide.
  misc_feature (10)..(11): N is any nucleotide.
  misc_feature (12)..(12): N is C or U.
  misc_feature (13)..(13): N is any nucleotide.
  misc_feature (14)..(14): N is C or U.
  misc_feature (15)..(17): N is any nucleotide.
  misc_feature (18)..(18): N is C or U.
  misc_feature (23)..(23): N is C or U.
  misc_feature (24)..(24): N is any nucleotide.
  misc_feature (28)..(28): N is A or G.
  misc_feature (32)..(32): N is C or U.
  misc_feature (47)..(48): N is C or U.
  misc_feature (52)..(52): N is A or G.
  misc_feature (62)..(62): N is A or G.